(19) United States
(12) Patent Application Publication
(10) Pub. No.: US 2025/0012797 A1
(43) Pub. Date: Jan. 9, 2025

(54) METHOD FOR DETECTING MICROORGANISMS AND USES THEREOF

(71) Applicants: Anton PERNTHANER, Palmerston North (NZ); Suzanne HURST, Palmerston North (NZ); David FLOSSDORF, Palmerston North (NZ); KORU DIAGNOSTICS LIMITED, Palmerston North (NZ)

(72) Inventors: Anton PERNTHANER, Palmerston North (NZ); Suzanne HURST, Palmerston North (NZ); David FLOSSDORF, Palmerston North (NZ)

(73) Assignee: KORU DIAGNOSTICS LIMITED, Palmerston North (NZ)

(21) Appl. No.: 18/707,237

(22) PCT Filed: Nov. 4, 2022
(86) PCT No.: PCT/NZ2022/050136

§ 371 Date: May 3, 2024

(30) Foreign Application Priority Data
Nov. 5, 2021 (NZ) 781706

(51) Int. Cl. G01N 33/569; G01N 33/53; G01N 33/543

(52) U.S. Cl. CPC G01N 33/56911; G01N 33/5306; G01N 33/54366; G01N 2333/245; G01N 2333/26; G01N 2333/285; G01N 2333/77; G01N 2400/50; G01N 2800/365

(57) ABSTRACT

The invention relates to methods of screening biological samples from a subject for the presence of one or more microorganisms, such as the presence in the subject of one or more pathogenic or potentially pathogenic microorganisms, and diagnostic kits and devices suitable for carrying out said methods. More particularly, the present invention relates to sensitive and specific methods for determining the presence of gram-negative bacteria or analytes therefrom in a sample from a subject using immunoassays, and devices and kits for carrying out such methods.

### TECHNICAL FIELD

The invention relates to methods of screening biological samples from a subject for the presence of one or more microorganisms, such as the presence in the subject of one or more pathogenic or potentially pathogenic microorganisms. More particularly, the present invention relates to sensitive and specific methods for determining the presence of gram-negative bacteria or analytes therefrom in a sample from a subject using immunoassays.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art. Any discussion of the prior art throughout the specification should in no way be considered as an admission that such prior art is widely known or forms part of the common general knowledge in the field or in any particular jurisdiction.

It is well recognised that pathogenic microorganisms are causative of and/or associated with a wide variety of diseases and conditions and have a very substantial impact on both health and wellbeing, and on economic activities.

Gram-negative bacteria are causative of a number of important infections, each of which is frequently exacerbated by the release of endotoxins from the bacterial membrane. Pneumonia, peritonitis, urinary tract infections, sepsis and bloodstream infections, persistent infections of wounds or surgical sites, and meningitis, are just some of the serious infections caused by gram-negative bacteria, such as but not limited to Escherichia coli, Serratia marcescens, Enterobacter cloacae, Salmonella spp., Pseudomonas aeruginosa, Proteus vulgaris and Klebsiella pneumoniae.

Rapid and sensitive detection of bacterial pathogens is of particular importance with the increasing prevalence of multidrug resistance in bacterial pathogens of humans, and of other animal species. Multidrug resistant gram-negative bacteria, such as various Enterobacteriaceae, are particularly problematic, as there is a paucity of effective treatment agents for these organisms.

Another example of an important condition primarily caused by bacterial infection is mastitis, an inflammation of the mammary gland typically associated with intramammary infection. While mastitis is problematic in a number of mammalian species, including humans, the diagnosis and treatment of mastitis is of significant importance to the dairy industry. Indeed, mastitis is the costliest disease in dairy cattle. In large part, this cost is associated with the need to discard milk from cows undergoing treatment for mastitis for which a withholding period in which milk cannot be collected for consumption pertains, together with reduced milk production, including amongst cows with asymptomatic or subclinical mastitis. The cost of veterinary care of infected cows, and labour costs, are significant. Animal wellbeing is also a serious consideration.

Important gram-negative pathogens associated with mastitis in cows include coliform bacteria such as E. coli, Enterobacter spp., Klebsiella spp., and Citrobacter.

Accordingly, the availability of robust, timely and accurate methods of detecting the presence of pathogenic microorganisms is of critical importance. For example, the robust and sensitive detection of pathogens associated with mastitis is of significant importance to agricultural industries. There is a need to develop new and improved methods for detecting gram-negative bacteria including those causative of or associated with a disease or condition, including methods that have high sensitivity and specificity, and/or avoid one or more of the disadvantages of existing methods, such as the need for aseptic sampling and processing, and time delays in obtaining results.

Similarly, there remains a need for methods of detecting pathogenic microorganisms that are capable of utilising readily available biological samples. For example, the use of milk samples in immunoassays is inherently difficult because milk contains cells, fat, protein and many other components that generally reduce the effectiveness of the assay. Furthermore, mastitic milk has highly elevated somatic cell counts (SCC) and may contain debris and bacterial conglomerates that complicate handling and reduce assay accuracy.

It is therefore an object of the invention to provide one or more diagnostically useful methods of detecting the presence of gram-negative bacteria in a sample or in a subject from which a sample is obtained, or to at least provide a useful alternative to existing detection methods, or to at least provide the public with a useful choice.

### SUMMARY OF THE INVENTION

In a first aspect, the invention relates to a method of determining the presence of a lipopolysaccharide analyte in a biological sample, the method comprising the steps of:
(a) a lipopolysaccharide analyte capture step, comprising contacting the sample with at least one capture reagent in the presence of a solid substrate, said capture reagent capable of binding to lipopolysaccharide;
(b) contacting the sample with a multimerizing reagent capable of binding to lipopolysaccharide, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with a binding reagent capable of binding to the complex comprising lipopolysaccharide analyte and multimeric multimerizing reagent;
(e) detecting the presence of the complex so formed;
   and wherein the presence of the complex is indicative of the presence of a lipopolysaccharide analyte in the sample.

In another aspect, the invention relates to a method of determining the presence of gram-negative bacteria in a sample or in a subject from whom a sample has been obtained, the method comprising
(a) a lipopolysaccharide analyte capture step, comprising contacting the sample with at least one capture reagent in the presence of a solid substrate, said capture reagent capable of binding to lipopolysaccharide;
(b) contacting the sample with a multimerizing reagent capable of binding to lipopolysaccharide, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with binding reagent capable of binding to the complex comprising lipopolysaccharide analyte and multimeric multimerizing reagent;
(e) detecting the presence of the complex so formed;
   wherein the presence of the complex is indicative of the presence of gram-negative bacteria in the sample or in the subject from whom the sample was obtained.

In a further aspect, the invention relates to a method of diagnosing mastitis in a subject or of identifying a subject at increased risk of having or developing mastitis, the method comprising the steps:
(a) providing a sample from the subject;
(b) a lipopolysaccharide analyte capture step, comprising contacting the sample with at least one capture reagent in the presence of a solid substrate, said capture reagent capable of binding to lipopolysaccharide;
(c) contacting the sample with a multimerizing reagent capable of binding to lipopolysaccharide, wherein when contacted with the sample said multimerizing reagent is capable of further multimerization;
(d) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(e) optionally contacting the sample with binding reagent capable of binding to the complex comprising lipopolysaccharide analyte and multimeric multimerizing reagent;
(f) detecting the presence of the complex so formed;
   wherein the presence of the complex indicates that the subject has mastitis or is at increased risk of developing mastitits.

In a further aspect, the invention relates to a method of diagnosing a disease or condition in a subject or of identifying a subject at increased risk of having or developing a disease or condition, wherein the disease or condition is caused by or associated with the presence of a gram-negative bacteria, the method comprising the steps:
(a) providing a sample from the subject;
(b) a lipopolysaccharide analyte capture step, comprising contacting the sample with at least one capture reagent in the presence of a solid substrate, said capture reagent capable of binding to lipopolysaccharide;
(c) contacting the sample with a multimerizing reagent capable of binding to lipopolysaccharide, wherein when contacted with the sample said multimerizing reagent is capable of further multimerization;
(d) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(e) optionally contacting the sample with binding reagent capable of binding to the complex comprising lipopolysaccharide analyte and multimeric multimerizing reagent;
(f) detecting the presence of the complex so formed;
   wherein the presence of the complex indicates that the subject has the disease or condition or is at increased risk of developing the disease or condition.

In various embodiments, the disease or condition is selected from the group consisting of Brucellosis; Campylobacter infections; Cholera; Escherichia coli (E. coli) infections; Haemophilus influenzae infections; Klebsiella infections; Proteus infections, Legionellosis, including Legionnaires' disease; Pertussis; Plague; Pseudomonas infections; Salmonella infections; sepsis, septic shock, Shigellosis; Tularemia; Porphyromonas gingivalis infections, Heligobacter infections and Typhoid fever

It will be appreciated that the absence of the complex is indicative of the absence of a biologically relevant lipopolysaccharide analyte in the biological sample, and/or is indicative of the absence of gram-negative bacteria in the biological sample or in the subject from whom the biological sample was obtained.

In a further aspect the invention relates to a method of determining the presence of a lipopolysaccharide analyte in a biological sample or of determining the presence of gram-negative bacteria in a sample or in a subject from whom a sample has been obtained, the method comprising the steps of:
(a) a lipopolysaccharide analyte capture step, comprising contacting the sample with at least one capture reagent, said capture reagent capable of binding to lipopolysaccharide or to a complex in which lipopolysaccharide is present;
(b) contacting the sample with a multimerizing reagent capable of binding to lipopolysaccharide or a complex in which lipopolysaccharide is present, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with a binding reagent capable of binding to the complex comprising lipopolysaccharide analyte and multimeric multimerizing reagent;
(e) detecting the presence of the complex so formed;
   and wherein the presence of the complex is indicative of the presence of a lipopolysaccharide analyte in the sample or is indicative of the presence of gram-negative bacteria in the sample or in the subject from whom the sample has been obtained.

In another aspect the invention relates to a method of determining the presence of a lipopolysaccharide analyte in a biological sample or of determining the presence of gram-negative bacteria in a sample or in a subject from whom a sample has been obtained, the method comprising the steps of:
(a) contacting the sample with at least one capture reagent and at least one multimerizing reagent, said capture reagent capable of binding to lipopolysaccharide or to a complex in which lipopolysaccharide is present, and said multimerizing reagent capable of binding to lipopolysaccharide or a complex in which lipopolysaccharide is present, wherein when contacted with the sample said multimerizing reagent is capable of further multimerization;
(b) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(c) optionally contacting the sample with a binding reagent capable of binding to the complex comprising lipopolysaccharide analyte and multimeric multimerizing reagent;
(d) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In another aspect the invention relates to a method of determining the presence of a lipopolysaccharide analyte in a biological sample or of determining the presence of gram-negative bacteria in a sample or in a subject from whom a sample has been obtained, the method comprising the steps of:
(a) contacting the sample with a capture reagent comprising Polymyxin B, and at least one multimerizing reagent, said multimerizing reagent capable of binding to lipopolysaccharide or a complex in which lipopolysaccharide is present, wherein when contacted with the sample said multimerizing reagent is capable of further multimerization;
(b) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(c) contacting the sample with a binding reagent capable of binding to the complex comprising lipopolysaccharide analyte and multimeric multimerizing reagent;
(d) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In one embodiment, the complex comprising lipopolysaccharide comprises one or more lipopolysaccharide binding proteins, such as one or more antibodies. In one example, the biological sample comprises LPS wherein at least a portion of the LPS present in the sample is bound by one or more antibodies. For example, at least a portion of the LPS is present in an LPS/antibody complex.

In a further aspect the invention relates to a method of determining the presence of a lipopolysaccharide analyte in a biological sample or of determining the presence of gram-negative bacteria in a sample or in a subject from whom a sample has been obtained, the method comprising the steps of:
(a) a lipopolysaccharide analyte capture step, comprising contacting the sample with at least one capture reagent, said capture reagent capable of binding to lipopolysaccharide or to a complex in which lipopolysaccharide is present;
(b) maintaining the lipopolysaccharide analyte and the capture reagent in contact for a time and under conditions suitable for the formation of a complex comprising lipopolysaccharide analyte and capture reagent;
(c) optionally contacting the sample with an albumin, such as bovine serum albumin, or an ovalbumin;
(d) optionally contacting the sample with a binding reagent capable of binding to the complex comprising lipopolysaccharide analyte and capture reagent;
(e) contacting the sample with an immunoglobulin binding protein;
(f) detecting the presence of the complex so formed;
   and wherein the presence of the complex is indicative of the presence of a lipopolysaccharide analyte in the sample or is indicative of the presence of gram-negative bacteria in the sample or in the subject from whom the sample has been obtained.

In various embodiments, the lipopolysaccharide capture step is done in the presence of one or more non-ionic detergents. For example, the sample is contacted with the at least one capture reagent in the presence of one or more non-ionic detergents. In certain examples, the method comprises the preliminary step of contacting the sample with one or more non-ionic detergents prior to contact with the one or more capture reagents.

In various embodiments, the immunoglobulin binding protein is selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof.

In various embodiments, the immunoglobulin binding protein is detectably labelled.

In another aspect the invention relates to a method of determining the presence of a lipopolysaccharide analyte in a biological sample or of determining the presence of gram-negative bacteria in a sample or in a subject from whom a sample has been obtained, the method comprising the steps of:
(a) contacting the sample with a capture reagent, said capture reagent comprising Polymyxin B and one or more polyethylene glycols, wherein said capture reagent is capable of binding to lipopolysaccharide or a complex in which lipopolysaccharide is present;
(b) maintaining the lipopolysaccharide analyte and the capture reagent in contact for a time and under conditions suitable for formation of a complex comprising lipopolysaccharide analyte and capture reagent;
(c) contacting the sample with a binding reagent capable of binding to the complex comprising lipopolysaccharide analyte and capture reagent;
(d) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In one embodiment, the complex comprising lipopolysaccharide comprises one or more lipopolysaccharide binding proteins, such as one or more antibodies. In one example, the biological sample comprises LPS wherein at least a portion of the LPS present in the sample is bound by one or more antibodies. For example, at least a portion of the LPS is present in an LPS/antibody complex.

In one example, the method comprises:
(a) contacting the sample with a capture reagent, said capture reagent comprising Polymyxin B and PEG20, wherein said capture reagent is capable of binding to lipopolysaccharide or a complex in which lipopolysaccharide is present;
(b) maintaining the lipopolysaccharide analyte and the capture reagent in contact for a time and under conditions suitable for formation of a complex comprising lipopolysaccharide analyte and capture reagent;
(c) contacting the sample with a binding reagent capable of binding to the complex comprising lipopolysaccharide analyte and capture reagent, said binding reagent comprising a labelled immunoglobulin binding protein selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof;
(d) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In another example the method comprises:
(a) contacting the sample with a capture reagent, said capture reagent comprising Polymyxin B and one or more polyethylene glycols, such as PEG20, wherein said capture reagent is capable of binding to lipopolysaccharide or a complex in which lipopolysaccharide is present;
(b) maintaining the lipopolysaccharide analyte and the capture reagent in contact for a time and under conditions suitable for formation of a complex comprising lipopolysaccharide analyte and capture reagent;
(c) optionally contacting the sample with bovine serum albumin;
(d) optionally contacting the sample with a binding reagent capable of binding to the complex comprising lipopolysaccharide analyte and capture reagent, said binding reagent comprising one or more antibodies,
(e) contacting the sample with an immunoglobulin binding protein, such as a labelled immunoglobulin protein, including an immunoglobulin protein selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof;
(f) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In another example, the method comprises:
(a) contacting the sample with a capture reagent, said capture reagent comprising Polymyxin B and one or more polyethylene glycols, such as PEG20, wherein said capture reagent is capable of binding to lipopolysaccharide or a complex in which lipopolysaccharide is present;
(b) maintaining the lipopolysaccharide analyte and the capture reagent in contact for a time and under conditions suitable for formation of a complex comprising lipopolysaccharide analyte and capture reagent;
(c) contacting the sample with bovine serum albumin;
(d) contacting the sample with a binding reagent capable of binding to the complex comprising lipopolysaccharide analyte and capture reagent, said binding reagent comprising one or more antibodies,
(e) contacting the sample with a labelled immunoglobulin binding protein selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof;
(f) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In another example, the method comprises:
(a) contacting the sample with one or more non-ionic detergents;
(b) contacting the sample with a capture reagent, said capture reagent comprising Polymyxin B and a polyethylene glycol, such as PEG20, wherein said capture reagent is capable of binding to lipopolysaccharide or a complex in which lipopolysaccharide is present;
(c) maintaining the lipopolysaccharide analyte and the capture reagent in contact for a time and under conditions suitable for formation of a complex comprising lipopolysaccharide analyte and capture reagent;
(d) contacting the sample with an albumin or ovalbumin, such as bovine serum albumin;
(e) optionally contacting the sample with a binding reagent capable of binding to the complex comprising lipopolysaccharide analyte and capture reagent, said binding reagent comprising one or more antibodies,
(f) contacting the sample with an immunoglobulin binding protein, such as a labelled immunoglobulin protein, including an immunoglobulin protein selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof;
(g) detecting the presence of the complex so formed;
and wherein the presence of the complex is indicative of the presence of a lipopolysaccharide analyte in the sample, or is indicative of the presence of gram-negative bacteria in the sample or in the subject from whom the sample has been obtained.

In a further example, the method comprises:
(a) contacting the sample with one or more non-ionic detergents, such as a polyoxyethylene sorbitol (for example polysorbate 20, polysorbate 40) or a polyethylene oxide (for example Triton);
(b) contacting the sample with a capture reagent, said capture reagent comprising Polymyxin B and a polyethylene glycol, such as PEG20, wherein said capture reagent is capable of binding to lipopolysaccharide or a complex in which lipopolysaccharide is present;
(c) maintaining the lipopolysaccharide analyte and the capture reagent in contact for a time and under conditions suitable for formation of a complex comprising lipopolysaccharide analyte and capture reagent;
(d) contacting the sample with an albumin or ovalbumin, such as bovine serum albumin;
(e) contacting the sample with a binding reagent capable of binding to the complex comprising lipopolysaccharide analyte and capture reagent, said binding reagent comprising one or more antibodies,
(f) contacting the sample with an immunoglobulin binding protein, such as a labelled immunoglobulin protein, including an immunoglobulin protein selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof;
(g) detecting the presence of the complex so formed;
and wherein the presence of the complex is indicative of the presence of a lipopolysaccharide analyte in the sample, or is indicative of the presence of gram-negative bacteria in the sample or in the subject from whom the sample has been obtained.

Any of the embodiments described herein can relate to any of the aspects presented herein.

In one embodiment, the complex comprising lipopolysaccharide analyte is detected using immunoassay. In various embodiments, the immunoassay is or includes one or more various formats, for example enzyme-linked immunosorbent assay (ELISA), Western blot, flow-through (vertical flow) test, or lateral flow assay.

In one embodiment, the complex comprising lipopolysaccharide analyte and multimeric multimerizing reagent is detected using immunoassay. In various embodiments, the immunoassay is or includes one or more various formats, for example enzyme-linked immunosorbent assay (ELISA), Western blot, flow-through (vertical flow) test, or lateral flow assay.

In various embodiments, the presence of the complex comprising lipopolysaccharide analyte is detected using one or more secondary antibodies, such as one or more species-specific secondary antibodies.

In various embodiments, the presence of the complex comprising lipopolysaccharide analyte and multimeric multimerizing reagent is detected using one or more secondary antibodies, such as one or more species-specific secondary antibodies.

In other embodiments, the presence of the complex comprising lipopolysaccharide analyte is detected using an immunoglobulin binding protein other than antibodies. Representative examples of such immunoglobulin binding proteins include, for example, Protein G, Protein A, a Protein A/G conjugate, a Protein G fusion protein, a Protein A fusion protein, a Protein A/G fusion protein, an immunoglobulin-binding fragment thereof, or any combination of two of more thereof. In one example, the presence of one or more antibodies is detected using Protein G, or a Protein A/G fusion protein.

In other embodiments, the presence of the complex comprising lipopolysaccharide analyte and multimeric multimerizing reagent is detected using an immunoglobulin binding protein other than antibodies. Representative examples of such immunoglobulin binding proteins include, for example, Protein G, Protein A, a Protein A/G conjugate, a Protein G fusion protein, a Protein A fusion protein, a Protein A/G fusion protein, an immunoglobulin-binding fragment thereof, or any combination of two of more thereof. In one example, the presence of one or more antibodies is detected using Protein G, or a Protein A/G fusion protein.

In one example, the subject is human.

In one example, the subject is bovine, caprine, or ovine.

In various embodiments, the sample comprises or is selected from the group consisting of milk, blood, serum, plasma, urine, saliva, cerebrospinal fluid, cervical or urethral fluid.

In one example, the sample comprises milk, such as milk or composite milk from a subject in early lactation. For example, the subject is at less than about 30 days' lactation, such as less than about 25 days' lactation, less than about 20 days' lactation, or less than about 15 days' lactation, or about 5 days' lactation.

In one example, the milk or composite milk is from a subject after about 14 days' lactation. For example, the milk or composite milk is from a subject at about 14 days to about 120 days' lactation, such as from about 14 days to about 90 days, from about 14 days to about 75 days, or from about 14 days to about 60 days.

In one example, the milk or composite milk is from a subject in late lactation. For example, the subject is at more than about 240 days' lactation, or more than about 270 days' lactation.

In certain embodiments, the milk or composite milk is from a subject within one month from the end of lactation, for example, to assess the need for treatment during and/or after the end of lactation.

In various embodiments, the at least one capture reagent is immobilised on the solid support.

In various embodiments, the lipopolysaccharide analyte in the sample is derived from the cell membrane of Gram-negative bacteria.

In various embodiments, the gram-negative bacteria from which the lipopolysaccharide is derived or the presence of which is determined is a bacteria selected from the group consisting of Escherichia spp., Pasteurella spp., and Klebsiella spp.

In various embodiments, the sample is selected from the group consisting of milk, blood, serum, plasma, urine, saliva, cerebrospinal fluid, cervical or urethral fluid.

In various embodiments, at least one of the at least one capture reagent is selected from the group consisting of a lectin, a lipopolysaccharide binding protein, a lipopolysaccharide binding compound, and an antibiotic.

In one example, the antibiotic is selected from the group consisting of cyclic peptide-comprising antibiotics.

In one example, the at least one capture reagent comprises Polymyxin B.

In one example, the at least one capture reagent comprises a molecular crowding agent.

In one example, the at least one capture reagent comprises a polyethylene or a polyethylene glycol.

In one example, the polyethylene glycol is PEG20.

In one example, the at least one capture reagent comprises Polymyxin B and one or more agents selected from the group consisting of a polyethylene or a polyethlene glycol. In one specifically contemplated example, the capture reagent comprises Polymyxin B and PEG20.

In various embodiments, the multimerizing agent is selected from the group consisting of a lectin, a lipopolysaccharide binding protein, a lipopolysaccharide binding compound, and an antibiotic.

In one embodiment, the multimerizing agent is a lipopolysaccharide binding protein.

In various embodiments, the multimerizing agent is detectably labelled.

In various embodiments, the multimerizing agent is conjugated to a detection system.

In one example, the multimerizing agent is Concanavalin A.

In one example, the multimerizing agent is an albumin, such as bovine serum albumin.

In various embodiments, the binding reagent comprises an antibody or fragment thereof capable of selectively binding one or more components of the complex comprising lipopolysaccharide. In one example, the binding reagent comprises colostrum, milk, serum, or any combination thereof, comprising one or more antibodies.

In various embodiments, contacting the lipopolysaccharide analyte with the capture reagent is performed in the presence of one or more detergents. For example, contacting the lipopolysaccharide analyte with the capture reagent is done in a buffer comprising one or more detergents, such as one or more non-ionic detergents, for example a polysorbate such as a polyoxyethylene sorbitol (e.g. polysorbate 20, polysorbate 40) or a polyoxyethylene (20) sorbitan monoalkylate (the Tween series of detergents), a polyoxamer, or a polyethylene oxide such as a polyoxyethylene p-t-octyl phenol derivative (such as the Triton series of detergents such as Triton X-100, Triton X-102, Triton X-114). In certain specifically contemplated examples, the detergent is selected from the group consisting of Tween20™, Tween80™, Triton X-100™, and Triton X114™.

In various embodiments, the method comprises step (d), and wherein the binding reagent of step (d) is an antibody or fragment thereof capable of selectively binding one or more components of the complex comprising lipopolysaccharide analyte and multimeric multimerizing reagent.

In various embodiments, the method comprises step (d), and wherein the binding reagent of step (d) is an antibody or fragment thereof having improved binding to a component of the complex comprising lipopolysaccharide analyte and multimeric multimerizing reagent when said component is present in said complex compared to the binding of the binding reagent to the component of the complex when said component is not present in the complex.

In one embodiment, the method comprises the steps of:
(a) a lipopolysaccharide analyte capture step, comprising contacting the sample with a capture reagent comprising Polymyxin B in the presence of a solid substrate; and
(b) contacting the sample with a multimerizing reagent capable of binding to lipopolysaccharide, said multimerizing reagent comprising Concanavalin A and wherein when contacted with the sample at least some of the Concanavalin A is dimeric;
(c) maintaining the lipopolysaccharide analyte and the Concanavilin A in contact for a time and under conditions suitable for multimerization of the Concanavalin A and the formation of a complex comprising lipopolysaccharide analyte and multimeric Concanavalin A, said conditions comprising maintaining the sample at a pH of at least about 7.2;
(d) optionally contacting the sample with binding reagent capable of binding to the complex comprising lipopolysaccharide analyte and multimeric Concanavalin A;
(e) detecting the presence of the complex so formed;
   and wherein the presence of the complex is indicative of the presence of a lipopolysaccharide analyte in the sample, or is indicative of the presence of gram-negative bacteria in the sample or in the subject from whom the sample has been obtained.

In one embodiment, the method comprises the steps of:
(a) contacting the sample with at least one capture reagent comprising Polymyxin B and at least one multimerizing reagent, said multimerizing reagent capable of binding to lipopolysaccharide or a complex in which lipopolysaccharide is present, wherein when contacted with the sample said multimerizing reagent is capable of further multimerization; and
(b) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(c) optionally contacting the sample with binding reagent capable of binding to the complex comprising lipopolysaccharide analyte and multimeric multimerizing reagent;
(d) detecting the presence of the complex so formed;
   and wherein the presence of the complex is indicative of the presence of a lipopolysaccharide analyte in the sample or is indicative of the presence of gram-negative bacteria in the sample or in the subject from whom the sample has been obtained.

In one example, the multimerizing reagent comprises albumin, such as bovine serum albumin.

In one example, the multimerizing reagent comprises Concanavalin A, for example the multimerizing reagent comprises Concanavalin A and wherein when contacted with the sample at least some of the Concanavalin A is dimeric.

In one example, prior to contacting the sample in step (b) at least some of the Concanavalin A is maintained at or below pH 5.4.

In one example, contacting the sample with Concanavalin A comprises raising the pH at which the Concanavalin A is maintained.

In one example, contacting the sample with Concanavalin A comprises raising the pH at which the Concanavalin A is maintained to a pH of about 7.2 or above.

In various embodiments, the detection of the complex comprises contacting the sample with an antibody-binding reagent. In one example, the biological sample comprises LPS wherein at least a portion of the LPS present in the sample is bound by one or more antibodies, for example, at least a portion of the LPS is present in an LPS/antibody complex.

In various embodiments, the antibody-binding reagent is capable of binding one or more endogenous lipopolysaccharide-binding antibodies present in the sample.

In various embodiments, the method comprises step (d), and wherein the binding reagent of step (d) is an antibody or fragment thereof capable of selectively binding the complex comprising lipopolysaccharide analyte and multimeric Concanavalin A.

In one example, the binding reagent of step (d) is or comprises polyclonal antibodies, including labelled polyclonal antibodies.

In various embodiments, the presence of the binding reagent is determined by contacting the sample with a second binding reagent.

In one example, the second binding reagent is an antibody-binding reagent.

In one embodiment, at least one of the binding reagents is detectably labelled.

In various embodiments, the antibody-binding reagent is an antibody binding protein.

In one embodiment, the antibody-binding reagent is selected from the group consisting of: an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

In one example, the antibody-binding reagent comprises detectably labelled Protein A or a fragment thereof, and/or detectably labelled Protein G or a fragment thereof.

In one example, the presence of the complex comprising lipopolysaccharide and multimeric Concanavalin A is by detection of tetrameric Concanavalin A.

In one example, one or more Concanavalin A monomers or dimers present in the Concanavalin tetramer is labelled with a donor fluorophore, and one or more Concanavalin A monomers or dimers present in the tetramer is labelled with an acceptor fluorophore.

In various embodiments, the method comprises an ELISA.

Another aspect of the present invention relates to an analytical test device for detecting the presence of a lipopolysaccharide analyte in a liquid biological sample, the device comprising: a solid support having reversibly immobilised thereon in a first zone of the support a capture reagent capable of binding lipopolysaccharide, said capture reagent comprising Polymyxin B, optionally together with one or more molecular crowding reagents.

Another aspect of the present invention relates to an analytical test device for detecting the presence of a lipopolysaccharide analyte in a liquid biological sample, the device comprising:
(a) a solid support formed of a material enabling capillary flow of fluid along at least a portion of the support;
(b) a sample pad located proximal to one end of the solid support for receiving a sample of the fluid, wherein the sample pad comprises a filter membrane for removal of one or more components from the sample;
(c) a conjugate pad located on or in the solid support so that the sample flows under capillary action along the support from the sample pad to the conjugate pad and mobilises a conjugate contained in the conjugate pad, the conjugate comprising a multimerizing reagent capable of binding lipopolysaccharide, wherein said multimerizing reagent is bound to or capable of being bound by a detection agent;
(d) a detection band comprising a capture reagent immobilised on or in the support along a band located substantially perpendicular to the direction of flow of the sample along the support so that when the mobilised conjugate contacts the capture reagent in the detection band the presence of the lipopolysaccharide analyte in the sample is indicated by a visible colour change or detection of a fluorescence signal; and
(e) optionally a wicking pad for receiving and retaining sample after passing through the detection band.

Another aspect of the present invention relates to an analytical test device for detecting the presence of a lipopolysaccharide analyte in a liquid biological sample, the device comprising:
(a) a solid support formed of a material enabling capillary flow of liquid along at least a portion of the support;
(b) a sample pad located proximal to one end of the solid support for receiving a sample of the liquid sample, wherein the sample pad comprises one or more buffers, one or more blocking agents, and dimeric Concanavalin A;
(c) a conjugate pad located on or in the solid support so that the liquid sample flows under capillary action along the support from the sample pad to the conjugate pad, wherein the conjugate pad comprises detectably labelled antibody-binding reagent and a buffer system capable of raising the pH of the sample when present at the conjugate pad to at least about 7, wherein the conjugate pad optionally comprises immobilised LPS; and
(d) optionally a wicking pad for receiving and retaining sample after passing through the conjugate pad.

In various embodiments, the sample pad and/or the conjugate pad comprises a reagent selected from the group consisting of PMB, PEG, and immobilised LPS.

In various embodiments, the device comprises an indicator, said indicator comprising bovine IgG as a positive control.

In various embodiments, the detectable label comprises gold, such as gold particles, gold nanoparticles, or gold nanospheres.

Another aspect of the present invention relates to a diagnostic kit comprising:
(a) a capture reagent selected from the group consisting of a lectin, a lipopolysaccharide binding protein, a lipopolysaccharide binding compound, and an antibiotic,
(b) a multimerizing reagent capable of binding to lipopolysaccharide;
(c) optionally one or more binding reagents capable of binding to a complex comprising lipopolysaccharide analyte and multimeric multimerizing reagent; and/or
(d) one or more detectably labelled binding reagents capable of binding to a complex comprising lipopolysaccharide analyte and multimeric multimerizing reagent;
(e) optionally one or more reagents for detecting the presence of a labelled reagent;
(f) and instructions for the use of the kit in the detection of one or more lipopolysaccharide analytes in a sample or the presence of gram-negative bacteria in a sample or in a subject from whom a sample has been obtained.

In one embodiment, the diagnostic kit comprises a microtitre plate, wherein one or more wells comprises one or more reagents selected from the group consisting of at least one capture reagent, at least one multimerizing reagent, and at least one binding reagent.

In one example the diagnostic kit comprises a microtitre plate, wherein one or more wells of the microtitre plate comprises at least one capture reagent selected from the group consisting of a lectin, a lipopolysaccharide binding protein, a lipopolysaccharide binding compound, and an antibiotic.

In one example, the diagnostic kit comprises a microtitre plate, wherein one or more wells comprises a multimerizing agent selected from the group consisting of a lectin, a lipopolysaccharide binding protein, a lipopolysaccharide binding compound, and an antibiotic.

In one example, the diagnostic kit comprises a microtitre plate, wherein one or more wells comprises Concanavalin A, for example one or more wells comprise Concanavalin A as the multimerizing agent.

In one embodiment, the diagnostic reagent, diagnostic kit, or composition additionally comprises one or more of the following: one or more preservatives, one or more immunomodulatory agents or molecules.

In various embodiments, the capture reagent comprises Polymyxin B.

In various embodiments of the analytical test device or the diagnostic kit, the multimerizing reagent comprises Concanavalin A.

In various embodiments of the analytical test device or the diagnostic kit, the detectably labelled binding reagent capable of binding to a complex comprising lipopolysaccharide analyte and multimeric multimerizing reagent is selected from the group consisting of an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

Correspondingly, kits, microtitre plates, and the like comprising a composition comprising one or more reagents selected from the group consisting of at least one capture reagent, at least one multimerizing reagent, and a binding reagent are also contemplated.

The invention further relates to a method of preparing a diagnostic apparatus comprising a sample-receiving surface, the method comprising immobilising on said sample-receiving surface a composition comprising one or more reagents selected from the group consisting of at least one capture reagent, at least one multimerizing reagent, and at least one binding reagent, wherein said sample-receiving surface is capable of receiving a liquid sample.

In one embodiment, said apparatus is a microtitre plate.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). These are only examples of what is specifically intended and all possible combinations of numerical values between the lowest value and the highest value enumerated are to be considered to be expressly stated in this application in a similar manner.

Those skilled in the art will appreciate the meaning of various terms of degree used herein. For example, as used herein in the context of referring to an amount (e.g., “about 9%”), the term “about” represents an amount close to and including the stated amount that still performs a desired function or achieves a desired result, e.g. “about 9%” can include 9% and amounts close to 9% that still perform a desired function or achieve a desired result. For example, the term “about” can refer to an amount that is within less than 10% of, within less than 5% of, within less than 1% of, within less than 0.1% of, or within less than 0.01% of the stated amount. It is also intended that where the term “about” is used, for example with reference to a figure, concentration, amount, integer or value, the exact figure, concentration, amount, integer or value is also specifically contemplated.

Other objects, aspects, features and advantages of the present invention will become apparent from the description that follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The invention is exemplified in the following non limiting embodiments and with reference to the accompanying figures, in which:

FIG. 1 presents a graph showing LPS detection by PMB capture and Protein G-based detection in an acute milk sample diluted 1:100.

FIG. 2 presents a graph showing that sample incubation with LBP reduces the ability of PMB to bind to LPS by competing for lipid-A binding, establishing the specific binding of PMB to LPS.

FIG. 3 presents a graph showing the effect of pre-incubation of LPS with PMB to reduce binding to PMB-coated ELISA plates.

FIG. 4 presents a graph showing that the addition of exogeneous antibodies to LPS samples present in the acute E. coli mastitis sample #137 enables detection.

FIG. 5 presents a graph showing antibodies from colostrum pools broadly recognise LPS from various gram-negative bacteria, while differing to some degree in their ability to recognise LPS from various bacteria. Left panel, US sourced antibody preparation (μg/mL); right panel, NZ sourced antibody preparation (μg/mL).

FIG. 6 presents a graph showing that antibodies from a serum pool (black bars) recognise LPS with a lower sensitivity than colostrum (white bars).

FIG. 7 presents a graph showing sample pre-treatment with ConA increases the sensitivity of LPS detection in a dose-dependent manner.

FIG. 8 presents two graphs showing: (A) Co-incubation of LPS with ConA (25 μg/ml) reduces antibody-facilitated detection, and (B) LPS Capture by PMB followed by treatment with ConA increases detection.

FIG. 9 presents a graph showing the effect of ionic detergents (Triton X-100, solid lines; Tween 20, dashed lines) on the sensitivity of LPS detection in samples from a range of different gram-negative species.

FIG. 10 presents a graph showing assay sensitivity was improved when a mixture of PMB and PEG20 was used for microtitre plate coating when compared to a sequential coating with PMB followed by blocking with PEG20.

FIG. 11 presents a graph showing that the inclusion of ConA improves the detection of LPS/antibody complexes in three acute E. coli (#137, #165, #257) mastitis cases.

FIG. 12 presents a graph showing that the inclusion of ConA followed by addition of exogeneous colostrum antibodies enables detection of LPS from a large range of gram-negative mastitis pathogens.

FIG. 13 presents a graph showing that the inclusion of ConA treatment provides an assay of superior sensitivity to those in which SBA or CJL lectins were substituted for ConA.

FIG. 14 presents a graph showing the analytical specificity of the ELISA assay for LPS from mastitis and human sepsis pathogens.

FIG. 15 presents two graphs showing the specificity of the assay for a wide range of LPS from gram-negative human pathogens. FIG. 15A presents data showing the detection of LPS from E. coli K12, E. coli 0128.12, E. coli O55.B5, Proteus vulgaris, Salmonella enterica, Pseudomonas aeruginosa, Klebsiella pneumoniae. FIG. 15B presents data showing the detection of LPS from K. oxytoca, S. ficaria, S. marcescens and E. cloacae.

FIG. 16 presents a graph showing the efficacy of the assay in specifically detecting O-antigen deficient E. coli K12 LPS, and that the assay is highly specific to gram-negative bacteria.

FIG. 17 is a graph showing the effect on detection of LPS of different sample extraction buffers as described herein in Example 13.

FIG. 18 is a graph showing the effect of incorporating a BSA-based blocking step after sample addition on ELISA performance, as described herein in Example 13.

FIG. 19 is a graph showing the specificity of detection of shed E. coli 5077 LPS in bovine urine, as described herein in Example 13.

FIG. 20 is a graph showing the detection of E. coli 5077 LPS in urine samples from different animal species, as described herein in Example 14.

FIG. 21 presents three graphs showing the detection of LPS from different bacteria in canine urine samples as described herein in Example 15. FIG. 21a shows the detection of E. coli 5077 LPS in canine urine samples. FIG. 21b shows the detection of S. marcescens LPS in canine urine samples. FIG. 21c shows the detection of K. oxytoca LPS in canine urine samples.

FIG. 22 presents a phylogenetric tree of gram-negative bacteria suitable for detection as described herein, from Anzai Y, Kim H, Park J Y, Wakabayashi H, Oyaizu H. Phylogenetic affiliation of the pseudomonads based on 16S rRNA sequence. Int J Syst Evol Microbiol 50: 1563-1589; August 2000; International Journal of Systematic and Evolutionary Microbiology 50 Pt 4(4):1563-89; DOI:10.1099/00207713-50-4-1563.

FIG. 23 presents two graphs showing mastitis milk contains elevated levels of LPS-IgG complex which strongly correlates with inflammation. [A] presents a scatter plot of milk LPS-IgG complex levels measured by ELISA in healthy (∘) and coliform acute mastitis populations (●). Data is expressed as % maximum levels; the horizontal bar representing the median within each animal population and ***P<0.001 signifies statistical difference from healthy population. [B] is a regression plot between inflammatory biomarker (milk LDH activity [U/L]) and milk LPS-IgG complex levels (% maximum levels) from combined healthy and acute mastitis population, linear (dashed) line represents the goodness of fit.

FIG. 24 presents photographs of lateral flow format assays for LPS/IgG complexes present in mastitis milk samples, as described herein in Example 17. The lateral flow tests of mastitis positive samples are shown in the left panel, while mastitis negative and a non-LPS buffer control are presented in the right panel. Here, Protein G 40 nm nanogold conjugate was used as labelled binding reagent.

FIG. 25 presents a photograph of lateral flow format assays for LPS/IgG complexes present in mastitis milk samples, as described in Example 17. The samples (from left to right) are those identified in Table 1 herein, and in this case detection of LPS/IgG complexes was done using Protein A/G 150 nm nanosphere conjugate.

FIG. 26 is a graph presenting the results of analyses of samples from human sepsis patients, showing the specificity and sensitivity of the assay for a wide range of Gram-negative human sepsis pathogens, as described herein in Example 18.

FIG. 27 is a graph showing that sample treatment with Triton X-114 but not with other detergents enables detection of LPS from spiked plasma samples, as described herein in Example 19.

FIG. 28 is two graphs showing the detection of LPS from E. coli (top graph) or S. marcescens (bottom graph) in plasma samples is influenced by the detergent used, as described in Example 19.

FIG. 29 is a graph showing the effect of the inclusion of PEG at 0.25% in the PMB capture buffer, as described in Example 19 herein.

FIG. 30 is a graph showing that highly sensitive detection of LPS from the coliforms P. aeruginosa, K. oxytoca, S. marcescens and E. coli spiked into ovine plasma can achieved, as described in Example 20 herein.

### DETAILED DESCRIPTION

The present invention relates to methods for determining the presence of lipopolysaccharide analytes in samples obtained from subjects, and the determination of the presence of corresponding gram-negative bacteria in the subject from which the sample was obtained. Importantly, the methods enable the identification of subjects having gram-negative bacterial infections, including those at risk of developing a disease associated with such gram-negative bacterial infections.

The detection of lipopolysaccharides in samples has long been recognised as being indicative of a gram-negative bacterial infection. It will be appreciated that the principle of using analytes derived from a pathogen to detect the presence of that pathogen in a sample or in the subject from which the sample has been obtained can be employed for the detection of one or more diseases or conditions in which the pathogen plays a role.

The invention relates to the applicant's development of a rapid, sensitive, and effective assay for the testing of biological samples, such as bovine milk samples or human serum samples, in order to determine whether the subject from which the sample has been obtained has a gram-negative bacterial infection. The method is based on the use of a combination of assay reagents, for example including one or more multimerizing reagents, to improve assay accuracy and/or sensitivity to enable the detection and identification of bacterially derived analytes, and thereby the presence of the pathogen.

The lipopolysaccharide analytes targeted in the various methods disclosed herein can be detected using an immunological assay, such as ELISA, Western Blots, Lateral Flow tests and Biosensors. ELISA tests have the advantage of being comparatively fast and accurate and the tests are amenable to high throughput and automation. Lateral flow tests are advantageously employed where there is no expectation of having ready access to the laboratory equipment necessary for ELISA or other immunoassay methods, such as in workplace or public event screening and the like. The rapid provision of results from lateral flow tests also enable rapid diagnoses, which in turn can ensure better treatment decisions and/or health outcomes, and can frequently avoid the need for time-consuming or costly sample logistics and laboratory analysis.

### Selected Definitions

The term “and/or” can mean “and” or “or”.

The terms “antibody” and “immunoglobulin” or “Ig” are generally used interchangeably herein, and refer to a glycoprotein produced by the immune system in response to the presence of an antigen.

The terms “comprise”, “comprises”, and “comprising” as used in this specification and claims are not to be interpreted in an exclusive or exhaustive sense, and mean “consisting at least in part of”. When interpreting each statement in this specification that includes the term “comprise”, “comprises”, or “comprising”, features other than that or those prefaced by the term may also be present. Related terms such as “including”, “include” and “includes” are to be interpreted in the same manner.

The term “consisting essentially of” when used in this specification refers to the features stated and allows for the presence of other features that do not materially alter the basic characteristics of the features specified.

The term “consisting of” as used herein means the specified materials or steps of the claimed invention, excluding any element, step, or ingredient not specified in the claim. The term “antigen” means a molecule having distinct surface features or epitopes capable of stimulating a specific immune response. Antibodies (immunoglobulins) are produced by the immune system in response to exposure to antigens. Antigens maybe proteins, carbohydrates or lipids, although only protein antigens are usually classified as immunogens because carbohydrates and lipids cannot elicit an immune response on their own.

The term “Immunoglobulin A” or “IgA” means a subclass of Ig that has an important role in the immune function of mucosal surfaces, including in the context of particularly contemplated embodiments herein, the udder of bovine cows.

The term “Immunoglobulin G” or “IgG” means a subclass of Ig that is mostly found in circulating bodily fluids such as blood and lymph.

The term “Immunoglobulin M” or “IgM” means a subclass of Ig that is produced mainly in the spleen and is usually the first antibody to appear in response to initial exposure to an antigen.

A “subject” as used herein is an animal, usually a mammal, including a mammalian companion animal or a human. Representative companion animals include feline, equine, and canine.

Representative agricultural animals include bovine, ovine, caprine, cervine, and porcine. Specifically contemplated subjects are subjects which are used commercially to produce milk, such as bovine, ovine, and caprine subjects.

Those skilled in the art will recognise, on reading this description, that various uses of such methods in the detection of gram-negative bacteria, and particularly pathogenic gram-negative bacteria, and in the diagnosis or prognosis of one or more diseases associated with or caused by such bacteria, are provided.

Advantageously, the diagnosis of a disease or condition in a subject or the identification of a subject at increased risk of having or developing a disease or condition, wherein the disease or condition is caused by or associated with the presence of a gram-negative bacteria made in accordance with methods described herein will in certain embodiments be useful to inform a therapeutic regimen to treat (including to control, reverse, mitigate and/or prevent) such a disease or condition in the subject. Thus, methods of treating one or more of such diseases in a subject the presence of which has been diagnosed or prognosed using one or more of the aspects described herein are specifically contemplated.

Accordingly, in another aspect the invention relates to a method of diagnosing and treating a disease or condition in a subject, or of identifying and treating a subject at increased risk of having or developing a disease or condition, wherein the disease or condition is caused by or associated with the presence of a gram-negative bacteria, the method comprising:
(a) providing a sample from the subject;
(b) a lipopolysaccharide analyte capture step, comprising contacting the sample with at least one capture reagent in the presence of a solid substrate, said capture reagent capable of binding to lipopolysaccharide;
(c) contacting the sample with a multimerizing reagent capable of binding to lipopolysaccharide, wherein when contacted with the sample said multimerizing reagent is capable of further multimerization;
(d) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(e) optionally contacting the sample with binding reagent capable of binding to the complex comprising lipopolysaccharide analyte and multimeric multimerizing reagent;
(f) detecting the presence of the complex so formed;
   wherein the presence of the complex indicates that the subject has the disease or condition or is at increased risk of developing the disease or condition, and administering a therapeutic regimen to a subject so identified as having the disease or condition or being at increased risk of developing the disease or condition to treat said disease or condition.

In yet another aspect of the present invention there is provided a method of treating a disease or condition in a subject or of treating a subject at increased risk of having or developing a disease or condition, wherein the disease or condition is caused by or associated with the presence of a gram-negative bacteria, the method comprising:
(a) providing the results of a diagnostic or prognostic method as contemplated herein;
(b) determining whether a result indicative that the subject has the disease or condition or is at increased risk of developing the disease or condition exists, and identifying a subject accordingly; and
(c) administering a therapeutic regimen to a subject so identified to treat the disease or condition.

The therapeutic regimens administered in accordance with the methods of the present invention include, by way of illustration and example only, include the administration of one or more antibiotics effective to treat gram-negative bacterial infections. For example, the method comprises administration of one or more agents selected from the group consisting of aminoglycosides, such as gentamicin, amikacin; beta-lactams; carbapenems, including imipenem, meropenem; cephalosporins, including cefotaxime, ceftazidime; chloramphenicols; fluorquinolones, such as ciprofloxacin, delafloxacin; fosfomycin; penicillins; polymyxins, such as colistin, polymyxin B; glycylcycline, such as tigecycline; sulphonamides, such as co-trimoxazole; tetracyclines, including doxycycline, eravacycline, minocycline, omadacycline; and ureidopenicillins, such as piperacillin; and any combination of two or more thereof, including combinations such as ceftolozane/tazobactam, ceftazidime/avibactam, meropenem/vaborbactam, and imipenem/cilastatin/relebactam.

### Gram-Negative Bacterial and Bacterial Pathogens

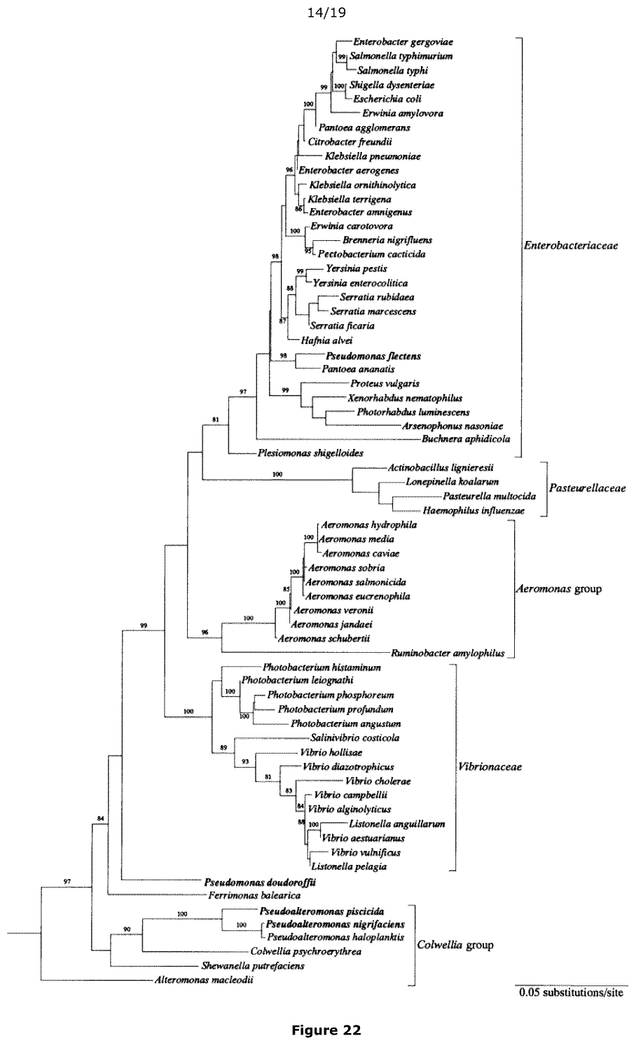

Gram-negative bacteria present one of the world's most significant public health problems due to their high resistance to antibiotics. These microorganisms have significant clinical importance in hospitals because they put patients present in the intensive care unit (ICU) at high risk and lead to high morbidity and mortality. Two large groups of gram-negative bacteria, the Enterobacteriaceae and the non-fermenters, are responsible for most clinically relevant isolates. Other gram-negative organisms of clinical relevance exist, including but not limited to Neisseria, Haemophilus spp., Helicobacter pylori, and Chlamydia trachomatis. A phylogenetic tree of gram-negative bacteria suitable for detection, including the Enterobacteriaceae, Pasteurellaceae and Aeromonas groups, using the methods and compositions described herein is presented in FIG. 22.

### Enterobacteriaceae

Enterobacteriaceae are a heterogeneous group widely dispersed in nature. They account for about 80% of gram-negative isolates with a myriad of disease-causing species in humans and other animals, including urinary tract infections, pneumonia, diarrhea, meningitis, sepsis, endotoxic shock, and many others. Species of particular concern and which frequently affect humans include Escherichia, Proteus, Enterobacter, Klebsiella, Citrobacter, Yersinia, Shigella, and Salmonella, among others.

Characteristics of Enterobacteriaceae typically employed in laboratory characterization include their being bacilli, non-sporulated, and having variable motility, are able to grow in the presence and absence of oxygen, are able to ferment glucose, are cytochrome oxidase negative, and can reduce nitrate to nitrite.

### Non-Fermenters

The non-fermenter gram-negative bacilli are usually encountered in the clinic than Enterobacteriaceae. However, they are clinically relevant since they cause severe, fatal infections, especially in a hospital environment. They also cause opportunistic diseases in ICU patients who undergo invasive procedures. The main non-fermenter gram-negative microorganisms that cause human disease are Pseudomonas aeruginosa, Acinetobacter baumannii, Burkholderia cepacia, Burkholderia pseudomallei, Stenotrophomonas spp., Alcaligenes spp., and Moraxella spp. These are characterised by being aerobic and non-sporulated, are incapable of fermenting sugars and instead use sugars via oxidative metabolism.

The assay methods and related aspects are useful in the detection of gram-negative bacteria, and finds particular application in the detection of gram-negative bacterial pathogens and as a result the diagnosis or prognosis of gram-negative bacterial infections.

Gram-negative bacteria can cause serious infections and are able to reach almost all systems in the subject organism, including the digestive system, nervous system, urinary system, and bloodstream. These microorganisms readily colonize the intestines, airways, and skin, thereby favouring the spread to other parts of the organism, especially in immunocompromised individuals. Gram-negative bacteria cause infections including pneumonia, peritonitis (inflammation of the membrane that lines the abdominal cavity), urinary tract infections, bloodstream infections including sepsis, wound or surgical site infections, and meningitis.

Nosocomial infections of the lower respiratory tract are particularly challenging to treat. This is because the pathogenic gram-negative bacteria typically involved are not only responsible for a good portion of these infections, they are non-responsive to antibiotic therapy due to the high resistance rates and the poor penetration of drugs into the lung parenchyma.

Another major concern is gastroenteritis caused by Enterobacteriaceae, particularly Shigella spp., Salmonella spp., and enteropathogenic E. coli. Gastroenteritis affects millions of people worldwide and is generally related to a lack of sanitation. Bacterial meningitis—a potentially fatal disease if not treated in time—is likewise a major concern both in the community and in the hospital environment. Urinary tract infections are also common, especially in young women. However, these infections became a problem with the widespread emergence of multi-resistant bacteria. Bacteremia is an important complication of these infections because of the acquisition of resistance.

Gram-negative pathogenic bacterial infections suitable for detection using the methods and related aspects disclosed herein include the following: Brucellosis; Campylobacter infections; Cholera; Escherichia coli (E. coli) infections; Haemophilus influenzae infections; Klebsiella infections; Proteus infections, Legionellosis, including Legionnaires' disease; Pertussis; Plague; Pseudomonas infections; Salmonella infections; Shigellosis; Tularemia; Porphyromonas gingivalis infections, Heligobacter infections and Typhoid fever.

Certain gram-negative bacteria of particular clinical concern are discussed below.

Brucella infections, frequently referred to as Brucellosis, are sometimes asymptomatic but most commonly result in acute illness, with symptoms including fever, arthralgia, headache, malaise, anorexia, constipation, respiratory tract symptoms and hepatosplenomegaly observed. If not adequately treated, chronic and persistent infections in joints, bone, liver or spleen can result. Brucellosis is often seen in New Zealand in those working with livestock, while internationally the ingestion of unpasteurised goat's cheese is the most common risk factor.

Campylobacter bacteria, usually Campylobacter jejuni, cause inflammation of the colon (colitis) that results in fever and diarrhoea. These bacteria are a common cause of infectious diarrhoea, and are notifiable diseases/diseases of public health interest in a number of countries.

In many countries, all strains of Vibrio cholerae (the causative pathogen of Cholera) are notifiable despite only isolates 01 and 0139 having the potential to produce cholera toxin being associated with clinical cholera. Cholera is characterised by vomiting and potentially severe diarrhoea, which may lead to profound dehydration and death. Methods to rapidly and sensitively detect Vibrio cholera are thus of significant interest, particularly in countries in which this pathogen is endemic or widespread.

Many strains of E. coli are harmless and/or non-pathogenic, being part of the normal gut microflora of mammals including humans. However, pathogenic strains of E. coli are frequently the causative agent of enteric disease and associated diarrhoea or dysentery, while other pathotypes cause extra-intestinal infections such as urinary tract infections or meningitis.

Legionella bacteria are ubiquitous in many environments, particularly in soil and aquatic environments, and Legionella infections are a common cause of pneumonia. Most cases in New Zealand are caused by L. longbeachae and L. pneumophila. Legionellosis is more common in older people, smokers, chronic disease sufferers and the immunocompromised. The most common clinical manifestation of Legionellosis reported worldwide is Legionnaires' disease, but non-pneumonic disease (such as Pontiac fever, an acute febrile illness usually accompanied by cough), and extrapulmonary disease, which usually manifest as infection of the skin, joints, pericardium or other organs, are thought to be often clinically un- or mis-diagnosed, and thus may be underreported.

Sepsis and septic shock are life-threatening conditions caused by one or more unregulated host responses to infection. Pathogenic gram-negative bacteria, including E. coli, Klebsiella pneumoniae, Acinetobacter baumannii, Enterobacter cloaceae, Proteus mirabilis, and Pseudominas aeruginosa, are common causative agents of sepsis and septic shock. Sepsis most commonly occurs as a result of infections in the urinary tract, the lungs, or the peritoneum, but infections in other organs including skin, the soft tissue, and the CNS are observed. Septic shock is usually considered to be a subset of sepsis in which profound abnormalities in the circulatory system, in cellular processes or in metabolic pathways are observed and are associated with an increased risk of mortality.

In principle, any source of LPS is amenable to detection using the methods and associated aspects disclosed herein, although the identification of pathogenic bacteria such as those discussed above is a focus of the present invention.

Particularly contemplated herein are detection and diagnostic methods, compositions, reagents, and kits that utilise a combination of the assay reagents described herein.

Accordingly, in one aspect the invention relates to a method of determining the presence of a lipopolysaccharide analyte in a biological sample, the method comprising the steps of:
(a) a lipopolysaccharide analyte capture step, comprising contacting the sample with at least one capture reagent in the presence of a solid substrate, said capture reagent capable of binding to lipopolysaccharide;
(b) contacting the sample with a multimerizing reagent capable of binding to lipopolysaccharide, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with a binding reagent capable of binding to the complex comprising lipopolysaccharide analyte and multimeric multimerizing reagent;
(e) detecting the presence of the complex so formed;
   and wherein the presence of the complex is indicative of the presence of a lipopolysaccharide analyte in the sample.

### Lipopolysaccharides

Lipopolysaccharides (LPS, often referred to as endotoxin) are carbohydrates present in the outer membrane of Gram-negative bacteria, generally comprising a 0-antigen, an inner oligosaccharide core, and an outer core. LPS has been reported to replicate septic shock when experimentally administered to mammals including humans, and has been reported to be a major factor in Gram-negative sepsis. Similarly, LPS has been reported to replicate symptoms of mastitis when experimentally administered.

The O-antigen is a repetitive glycan polymer attached to the core oligosaccharide and is the outermost component of LPS molecules. The composition of the O-antigen varies from species to species and strain to strain, with over 160 different O-antigen structures reported for E. coli strains alone. It has been reported that the O-antigen is the most variable portion of the LPS molecule, and most relevant to antigenic specificity.

The core oligosaccharide comprises sugars, commonly heptose and mannose derivatives such as 3-Deoxy-D-manno-oct-2-ulosonic acid (also known as keto-deoxyoctulosonate (KDO)), and often other non-carbohydrate components including amino acids or phosphates.

Lipid A is a hydrophobic fatty acid-containing molecule that anchors the LPS in the bacterial membrane. Typically, lipid A is a phosphorylated glucosamine disaccharide comprising multiple fatty acids. The particular composition of lipid A can differ across bacterial species and stains, though it is the most conserved component of LPS.

LPS has been reported to bind the the CD14/TLR4/MD2 receptor complex in many animal cell types. From a health perspective, the CD14/TLR4/MD2 receptor-positive cells of particular interest include monocytes, dendritic cells, macrophages, and B cells, the binding of LPS to which promotes the secretion of pro-inflammatory cytokines, nitric oxide, and eicosanoids.

In principle, any sample capable of comprising LPS is suitable for analysis using the methods and related aspects disclosed herein. Representative LPS-containing samples may comprise or be derived from milk, blood, serum, plasma, urine, saliva, cerebrospinal fluid, cervical or urethral fluid. In certain embodiments, tissue samples, including liquified tissue samples, may be used.

The at least one capture reagent binds to LPS or to LPS/antibody complexes present in the sample. In various embodiments of the assay methods and related aspects, the at least one capture reagent is immobilised on a solid support at which the various interactions employed in the assay take place. In certain embodiments, the at least one capture reagent is selected from the group consisting of a lectin, a lipopolysaccharide binding protein, a lipopolysaccharide binding compound, and an antibiotic.

In certain examples, the capture reagent is an antibiotic, such as a cyclic peptide-comprising antibiotic. In one particularly contemplated embodiment, the capture reagent comprises Polymyxin B.

In one embodiment, the capture reagent comprises a molecular crowding agent. In one example, the molecular crowding reagent is a polyethylene glycol.

The multimerizing reagent is capable of binding lipopolysaccharide or to LPS/antibody complexes present in the sample and/or captured by the at least one capture reagent. Without wishing to be bound by any theory, the inventors believe that multimerization of LPS-bound multimerizing reagent results in a configuration of LPS-containing complex that provides for enhanced binding and/or detection by binding reagents such as antibodies, whether endogeneous antibodies present in the sample, or exogeneous antibodies added to the assay.

In various embodiments, the multimerizing reagent is selected from the group consisting of a lectin, a lipopolysaccharide binding protein, a lipopolysaccharide binding compound, molecules that binds to the lipopolysaccharide-capture reagent complex, such as albumin or ovalbumin, and an antibiotic.

In certain embodiments, the multimerizing agent is a lipopolysaccharide binding protein, such as a detectably labelled lipopolysaccharide binding protein or bovine serum albumin.

In a particularly contemplated example, the multimerizing agent is Concanavalin A.

In one embodiment, the presence of an LPS analyte and/or complexes comprising an LPS analyte is detected, for example, using as a binding reagent an immunoglobulin, such as a species-specific, anti-Ig secondary antibody. In another example, the presence of an LPS analyte is detected using an immunoglobulin binding protein, such as Protein G.

In one embodiment, the presence of anti-LPS antibodies (and/or complexes comprising anti-LPS antibodies) is detected, for example, using species-specific, anti-Ig secondary antibodies. In another example, the presence of anti-LPS antibodies is detected using an immunoglobulin-binding protein, such as Protein G, Protein A, a Protein A/G conjugate, a Protein G fusion protein, a Protein A fusion protein, a Protein A/G fusion protein, an immunoglobulin-binding fragment thereof, or any combination of two of more thereof.

It will be appreciated that detection will typically involve the observation, whether directly or indirectly, of a detectable label, such as an enzymatic functionality conjugated to one of the assay reagents, such as the multimerizing reagent or a binding reagent, such as a detectably labelled immunoglobulin-binding moiety. Examples are well known in the art, and include, for example, antibodies and secondary antibodies and immunoglobulin-binding proteins conjugated to chromogenic reporters, which in the presence of a suitable substrate, catalyse the production of an observable colour change. Representative examples of labels include labels such as chemical substances such as biotin, digoxigenin (DIG), acridium ester, Flashlight and the like, enzymes such as horseradish peroxidase (HRP), ALP, glucose oxidase, 0 galactosidase and the like, fluorescent labels such as FITC, rhodamine, Cy3, Cy5, Texas Red, Alexa Fluors, BODIPYs, IRDyes, MFPs, Quantum Dots, AMCA, Allophycocyanin, BMP, Cy2, Cy3.5, Cy5.5, DTAF, DyLight 547, DyLight 647, FluoroNanogold, phycoerythrin, phycocyanin, R-PE, saporin, TRITC and the like, beads such as 60 μm Microbead, magnetic beads such as MagCellect Ferrofluid®, radioactive labels such as ¹²⁵I, gold particles, silver particles, latex particles, cellulose particles, gold or platnium nanoshells, agarose, FRET donor/quencher pairs, and the like.

Immunoassays are, in their most simple sense, binding assays where binding interactions are detected and/or quantified. As will be appreciated by those skilled in the art on reading this disclosure, specifically contemplated embodiments of immunoassays for practising the invention are the various types of enzyme linked immunosorbent assays (ELISAs) and/or radioimmunoassays (RIA) known in the art. However, it will be readily appreciated that detection of an LPS analyte and/or of anti-LPS antibodies in samples from a subject is not limited to such techniques, and/or western blotting, dot blotting, FACS analyses, and/or the like may also be used.

In one specifically contemplated exemplary ELISA, one or more capture reagents are immobilized onto a selected surface exhibiting protein affinity, such as a well in a polystyrene microtitre plate. Then, a sample from a subject is added to the wells, wherein the sample is capable of containing one or more LPS analytes or anti-LPS antibodies. After binding and/or washing to remove non-specifically bound agents, such as non-specifically-bound immune complexes, the bound analyte:capture reagent complex is detected. Detection is generally achieved by the addition of an antibody that is linked to a detectable label, or an immunoglobulin-binding protein linked to a detectable label, or in other embodiments is via a labelled multimerizing reagent.

The applicants have surprisingly found that a robust, highly specific assay can be performed as described herein using agents such as the lectin Concanavalin A. As described herein such agents are referred to herein as a multimerizing reagents, and without wishing to be bound by any theory, the applicants believe that such multimerizing agents, such as Concanavalin A, facilitates the recognition and/or binding of LPS by other binding reagents, such as antibodies. As shown in the Examples herein, the addition of Concanavalin A substantially enhances the sensitivity of LPS detection.

In an alternative embodiment, the applicants have surprisingly found that a robutst, highly specific assay can be performed as described herein (as shown, for example in Example 13 herein) using the albumin, bovine serum albumin. Here, as demonstrated in urine samples, the addition of bovine serum albumin enhances the sensitivity of LPS detection.

In an alternative exemplary ELISA, anti-LPS antibodies are immobilized onto a selected surface exhibiting protein affinity, such as a well in a polystyrene microtitre plate. Then, a sample from a subject is added to the wells. After binding and/or washing to remove non-specifically bound immune complexes, a multimerizing reagent is added, and the bound antibody:LPS complex is detected. Again, detection is generally achieved by the addition of an antibody that is linked to a detectable label, or an immunoglobulin-binding protein linked to a detectable label, or in other embodiments is via a labelled multimerizing reagent. This type of ELISA is often referred to as a simple “sandwich ELISA”.

Irrespective of the format employed, ELISAs have certain features in common, such as coating, incubating and binding, washing to remove non-specifically bound species, and detecting the bound complexes. These are described in general terms below, while a more detailed example of an ELISA procedure suitable for use in particular embodiments of the methods described is presented in the Examples herein.

To coat a plate with a particular reagent or assay component, such as a capture reagent, a blocking agent, an antigen, or an antibody, one will generally incubate the wells of the plate with a solution of the reagent or assay component, either overnight or for a specified period of hours. For example, in certain embodiments specifically contemplated herein, a capture reagent comprising Polymyxin B (PMB) and Polyethylene glycol (PEG) is added to one or more wells and incubated overnight.

In other embodiments, a mixture of capture reagent and blocking agentis added to one or more wells and incubated overnight. The wells of the plate will then be washed to remove incompletely adsorbed material. Any remaining available surfaces of the wells are then “coated” with a nonspecific protein that is antigenically neutral with regard to the test sample, or detection agent or sample. Generally, these blocking agents include bovine serum albumin (BSA), casein or solutions of milk powder, but as established in certain specifically contemplated embodiments exemplified herein, these coating and blocking steps can be combined rather than performed sequentially. The coating allows for blocking of nonspecific adsorption sites on the immobilizing surface and thus reduces the background caused by nonspecific binding of proteins, such as immunoglobulin-binding proteins or antisera, including those used for detection of LPS analyte-containing complexes, onto the surface.

In ELISAs, a secondary or tertiary detection agent is commonly used rather than a direct procedure. Thus, after binding of a protein or antibody to the well, coating with a non-reactive material to reduce background, and washing to remove unbound material, the immobilizing surface is contacted with the biological sample to be tested under conditions effective to allow binding and/or complex formation. Detection of the complex then requires a labelled secondary binding ligand, such as an immunoglobulin-binding agent or an antibody, or a secondary binding ligand or antibody in conjunction with a labelled tertiary antibody or a third binding ligand.

Following all incubation steps in an ELISA, the contacted surface is washed so as to remove non-complexed material. A representative washing procedure includes washing with a solution such as PBS/Tween, or borate buffer. Following the formation of specific complexes between the test sample and the originally bound material, and subsequent washing, the occurrence of even minute amounts of complexes may be determined.

The phrase “under conditions suitable for multimerization of the multimerizing reagent” as used herein include conditions in which the multimeric reagent is incubated in a buffered solution, typically one comprising BSA, bovine gamma globulin (BGG) or phosphate buffered saline (PBS) with or without Tween or another ionic detergent. These added agents also tend to assist in the reduction of nonspecific background. The phrase also contemplates conditions in which the incubation is at a temperature or for a period of time sufficient to allow effective binding and multimerization of the multimerizing reagent. Incubation steps are typically from about 30 minutes to 2 to 4 hours or so, at temperatures preferably in the order of 21° C. to 37° C., or may be overnight at about 4° C. or similar. In certain embodiments, such as embodiments in which the multimerizing reagent is Concanavalin A, conditions suitable for multimerization of the multimerizing reagent will typically include a pH of at least about 7, such as a pH of 7.2 or greater.

The phrase “under conditions suitable for the formation of a complex comprising lipopolysaccharide analyte and multimeric multimerizing reagent” as used herein include conditions in which the multimerizing reagent and the lipopolysaccharide are incubated in a buffered solution, typically one comprising BSA, bovine gamma globulin (BGG) or phosphate buffered saline (PBS) with or without Tween or another ionic detergent. As above, these added agents also tend to assist in the reduction of nonspecific background. The phrase also contemplates conditions in which the incubation is at a temperature or for a period of time sufficient to allow effective binding. Again, incubation steps are typically from about 30 minutes to 2 to 4 hours or so, at temperatures preferably in the order of 21° C. to 37° C., or may be overnight at about 4° C. or similar.

To provide for detection, an agent capable of binding to an LPS-containing complex is employed, whereby this detection agent has an associated label to allow detection. In most embodiments, the label is conjugated to a binding reagent capable of binding the complex, and typically this label will be an enzyme that will generate colour development upon incubating with an appropriate chromogenic substrate. Thus, for example, one will typically contact or incubate the LPS-containing complex(es) with, for example, a urease-, a glucose oxidase-, an alkaline phosphatase or a hydrogen peroxidase-conjugated antibody or immunoglobulin-binding protein for a period of time and under conditions that favour the development of further complex formation (e.g., incubation for 2 hours at room temperature in a PBS-containing solution such as PBS-Tween).

While many ELISA procedures employ antibodies, including species-specific antibodies, as the detection agent (frequently referred to as the secondary antibody), alternative agents capable of binding to LPS-containing complexes in which an antibody is present are also suitable for use. Specifically contemplated examples include immunoglobulin binding proteins such as Protein A, Protein G, and Protein A/G, each of which have been used extensively in immobilized form on chromatography supports for the purification of antibodies. While full length Protein A or Protein G can be used, in the context of antibody binding or purification a truncated recombinant form is typically used.

Protein A (SpA) in Staphylococcus aureus is encoded by the spa (staphylococcal protein A) gene. SpA is a 42 kDa protein comprising several regions with different functions: The signal sequence (S region) in the N-terminal part is followed by four or five highly homologous immunoglobulin G (IgG)-binding domains in tandem (the E, D, A, B, and C regions). The C-terminal region, also referred to as the X region, has two domains: (i) a repeat region XR, consisting of variable repeats with mostly octapeptide structures, and (ii) the XC region, consisting of a conserved sequence including an LPXTG-binding motif, which confers anchoring to the cell wall. It has been reported that SpA interacts with human IgG by binding to the Fc part of the immunoglobulin, and further that SpA can bind to other host structures, such as the von Willebrand factor to promote adhesion to platelets.

Protein G originates in group C and G Streptococcal bacteria. The native protein is a 56-kDa or 58-kDa polypeptide with multiple binding sites for immunoglobulins as well as a binding site for albumin.

The most commonly used recombinant form of the protein is a truncated version that has the albumin binding site removed but that retains the IgG-binding capabilities of the native molecule. Protein G binds to antibodies through the heavy chains in the region of the Fc fragment, but at a different site than that of Protein A. Protein A and Protein G exhibit different antibody binding specificities and affinities, and thus offer options in binding and purifying antibodies, depending on the type of antibody desired.

A chimeric fusion protein consisting of the combination of Protein A and Protein G, called Protein A/G, merges the advantages of both protein specificities into one molecule. As a consequence, Protein A/G is often the immunoglobulin binding protein of choice, and labelled examples have particular utility in the practice of the methods described herein.

Since most IgG type antibodies bind to Protein A, G, or A/G via their Fc fragments on the heavy chains, the antigen binding sites at the ends of the Fab fragments remain open to interact with antigens. As a consequence, detection of antigen-bound IgG antibodies (i.e., IgG antibody:antigen complexes) is readily achieved using these immunoglobulin binding proteins.

Detectably-labelled Protein G, Protein A, and Protein A/G, typically being recombinant forms of each, are readily available from commercial suppliers. For example, horseradish peroxidase conjugated Protein G (HRP-Protein G, cat #M00090) as used herein in the Examples was sourced from GenScript (NJ, USA), with HRP-Protein A (cat #M00089) also being available from this supplier. ThermoFisher Scientific supplies a range of recombinant Protein A/G and Protein G conjugates suitable for the detectable binding of antibodies, including peroxidase- or alkaline phosphatase-conjugated Protein A/G and Protein G, with biotinylated conjugates also being available from the same supplier, while fluorescein conjugated Protein G is available, for example from Rockland Immunochemicals Inc., PA, USA. It also is contemplated that the above reagents maybe packaged in a kit that may be produced commercially to measure the one or more LPS analytes, one or more LPS-specific antibodies, or LPS-containing complexes as described herein.

In one particularly contemplated embodiment, a diagnostic test kit based upon the detection of one or more LPS analytes, or of LPS-specific antibodies and therefore suitable for the detection of gram-negative bacterial infection, such as the diagnosis of mastitis or sepsis, comprises an ELISA test.

In one example of such a test, the walls of the wells of an ELISA plate are coated with one or more capture reagents as described herein, for example, a capture reagent selected from the group consisting of a lectin, a lipopolysaccharide binding protein, a lipopolysaccharide binding compound, and an antibiotic. After incubation with the test sample and other assay components including the multimerizing reagent (which may itself be detectably labelled) and optionally a binding reagent, detection by for example a colour reaction then reveals the presence of LPS-containing complexes and thus the presence of LPS analyte in the sample.

It will be appreciated that, when employed in the methods and associated aspects disclosed herein, secondary antibodies are advantageously species-specific antibodies. For example, when bovine samples are to be analysed, bovine anti-Ig antibodies will generally be used. In particularly contemplated embodiments herein, bovine milk samples are collected, and bovine anti-IgA and/or bovine anti-IgG secondary antibodies are used.

However, in certain embodiments, detection methods that utilize antibody-binding proteins other than antibodies are employed. Representative examples of other antibody binding proteins include, for example, Protein G, Protein A or Protein AG conjugates or fragments thereof. Methods for using such antibody-binding proteins are well established in the art and are exemplified herein in the Examples.

In particularly contemplated embodiments of the methods described herein, complex formation and/or detection is by immunoassay. The design of the immunoassay may vary. For example, the immunoassay may be based upon competition or direct reaction. Furthermore, protocols may use solid supports or may use cellular material. The detection of LPS-containing complexes will in certain embodiments involve the use of labelled antibodies such as labelled secondary antibodies, or labelled antibody-binding proteins, such as labelled Protein G. In various embodiments the labels may be, for example, enzymes, fluorescent-, chemoluminescent-, radioactive- or dye molecule labels.

The immunoassays contemplated herein include various formats, for example chip-based immunoassays, ELISA, Western blot, flow-through (vertical flow) tests, or lateral flow assays. Suitable methods for the detection of one or more LPS analytes in the sample include the enzyme-linked immunosorbent assay (ELISA), immunofluorescence test (IFT) and Western blot analysis. In one example, the method may be a flow-through (vertical flow) test or lateral flow dot dipstick test, and devices suitable for implementing such methods, such as a lateral flow device, are also contemplated.

It will be appreciated by those skilled in the art that certain of these methods and devices advantageously do not require sophisticated machinery to perform, and can be readily implemented at a point-of-care location, such as a milking shed, public health or school clinic, and the like.

Likewise, such methods directed to the identification of bacteria associated with production animals, such as milking bovine, ovine, or caprine, are amenable to implementation in real time, for example via in-flow deployment, allowing rapid identification of at risk subjects during milk collection whereby appropriate handling of the subject and/or precautionary measures, such as diversion of milk collected from an at-risk subject or from a subject determined to be suffering from mastitis or having asymptomatic or subclinical mastitis, can be taken.

Accordingly, the invention relates to a kit for screening a biological sample for the presence of one or more LPS analytes present in the biological sample. A representative kit contemplated herein comprises:
(a) a capture reagent selected from the group consisting of a lectin, a lipopolysaccharide binding protein, a lipopolysaccharide binding compound, and an antibiotic,
(b) a multimerizing reagent capable of binding to lipopolysaccharide;
(c) optionally one or more binding reagents capable of binding to a complex comprising lipopolysaccharide analyte and multimeric multimerizing reagent; and/or
(d) one or more detectably labelled binding reagent capable of binding to a complex comprising lipopolysaccharide analyte and multimeric multimerizing reagent;
(e) optionally one or more reagents for detecting the presence of a labelled reagent;
(f) and instructions for the use of the kit in the detection of one or more lipopolysaccharide analytes in a sample or the presence of gram-negative bacteria in a sample or in a subject from whom a sample has been obtained.

Notably, as established by the experimental data presented herein, certain embodiments of ELISA-based methods and lateral flow methods have surprising sensitivity, particularly when compared to other diagnostic methods.

Beneficially, this high sensitivity as observed in the analysis of bovine milk samples enables the detection of one or more LPS analytes in individual or pooled milk samples, including samples in which milk from each quarter of the udder has been pooled. Milk samples in which milk from each quarter of the udder, for example the udder of a bovine, a caprine, or an ovine subject, has been pooled is referred to herein as composite milk.

In some circumstances the use of composite milk samples can reduce the burden associated with testing, making sample collection substantially more straightforward, and testing more cost effective.

For example, the use of composite milk enables automation of sample collection, for example during regular herd testing or ‘milk recording’ procedures, such that an aliquot of composite milk from a given animal can be set aside for use in the methods described herein.

Similarly, as exemplified herein the methods described in this specification are amenable to use with other biological samples, including urine and plasma samples.

In a particularly contemplated embodiment, the kit is an ELISA kit, comprising one or more microtitre plates, one or more capture reagent and/or one or more compositions comprising one or more capture reagents, a multimerizing reagent and/or one or more compositions comprising one or more multimerizing reagents, and optionally one of more binding reagents and/or one or more compositions comprising one or more binding reagents. In certain examples, one or more of the compositions is a liquid composition suitable for administration to one or more of the microtitre plate wells at the end-user's discretion and direction. In other examples, one or more of the reagents or compositions have already been applied to one or more of the microtitre plate wells. For example, the kit comprises one or more microtitre plates comprising immobilised capture reagent, such as PMB.

In one particularly contemplated example, the kit is an ELISA kit, comprising one or more microtitre plates to which PMB in the presence of PEG, such as PEG20, has been applied. As shown in the examples provided herein, the use of a mixture of PMB+PEG20 surprisingly enables highly sensitive capture of one or more LPS analytes and thus highly sensitive ELISA analyses to be performed in accordance with this disclosure.

It will be appreciated that ELISA kits will typically include reagents, including secondary antibodies or immunoglobulin binding proteins or agents, particularly detectably labelled secondary antibodies or Ig-binding proteins, reporter enzyme substrate, buffers, and the like, together with instructions for use. Positive control and negative control samples are also frequently provided. For example, the kit will in certain embodiments include a reagent necessary for ELISA such as a sandwich method, a competition method, a direct adsorption method and the like, or a reagent necessary for conducting analysis such as a Western blotting method and the like. Generally, an instruction manual is added to the kit.

In another embodiment, the kit comprises a flow-through (vertical flow) test device or lateral flow assay device to detect the presence of one or more LPS analytes present in the biological sample.

In various embodiments, the device is a lateral flow device, such as a lateral flow test strip, or a dipstick test strip, or a dot test strip.

The diagnostic methods, kits, compositions and the like described herein usefully employ various interactions between the analyte to be detected, LPS, and other agents present in or added to the assay, to enable the sensitive detection of LPS in a sample, and thus the presence of gram-negative bacteria in the source from which the sample was obtained.

The invention is further described with reference to the following examples. It will be appreciated that the invention as claimed is not intended to be limited in any way by these examples.

### EXAMPLES

### Example 1: Assay Development for the Detection of LPS/Antibody Complex in Acute Mastitis Samples

This example presents experiments designed to explore the direct detection of LPS by ELISA.

Lipopolysaccharide (LPS) binding protein (LBP), Concanavalin A (ConA) and Polymyxin B (PMB) in various combinations were investigated as reagents that may be suitable for the detection of LPS in milk samples from cases of acute E. coli mastitis. Suitable conditions for reagent binding to ELISA plates were determined, leading to the successful immobilisation of LBP, ConA and PMB on ELISA plates.

The detection of LPS captured by immobilised PMB and detected by immobilised HRP-labelled anti-LPS antibody, was inconsistent and insufficiently reliable (data not shown).

An alternative method was developed, based on the hypothesis that LPS is present in the sample as an LPS/antibody complex, and that this complex may be detectable using Protein G-HRP.

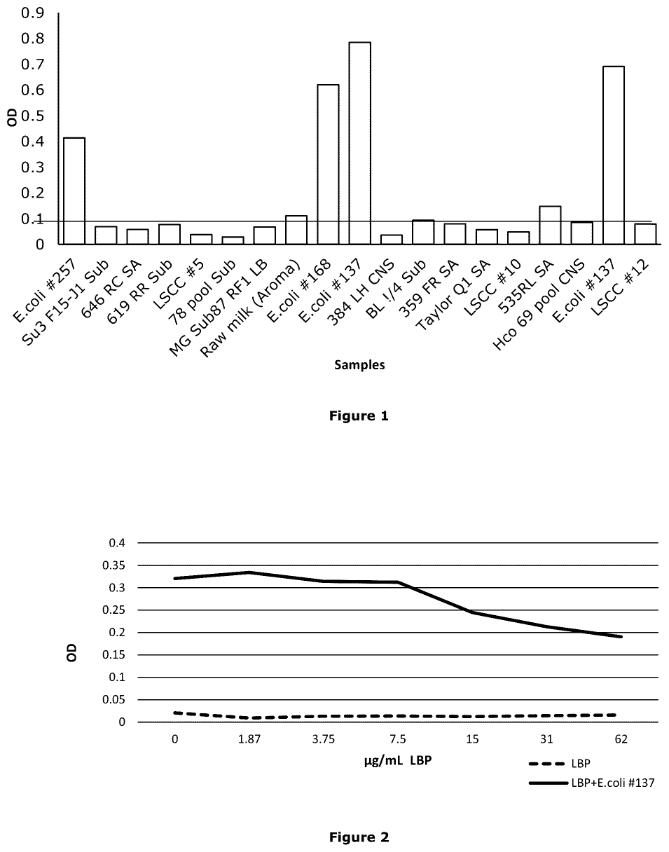

ELISA using PMB for the capture of LPS, and an optimal sample dilution system followed by Protein G-HRP based detection, was developed. This ELISA could detect LPS in E. coli-positive samples (FIG. 1), without any indication for cross-reactivity with other mastitis pathogens such as Staphylococcus aureus, coagulase negative Staphylococci (CNS) or Streptococcus uberis. These experiments established that host-produced antibodies present in LPS/antibody complexes can be used to detect LPS.

A cut-off was calculated based on the mean plus 3×STDEV of negative samples. Three acute E. coli samples were found to be positive while a range of other samples were negative.

The analytical specificity of the mastitis test was confirmed by pre-incubation of a sample with LBP. Pre-incubation with LBP was chosen because both PMB and LBP bind to lipid-A and therefore may compete for binding. As expected, sample treatment with LBP reduced LPS binding to PMB in a dose-dependent manner, thus demonstrating specificity of PMB binding to LPS (FIG. 2).

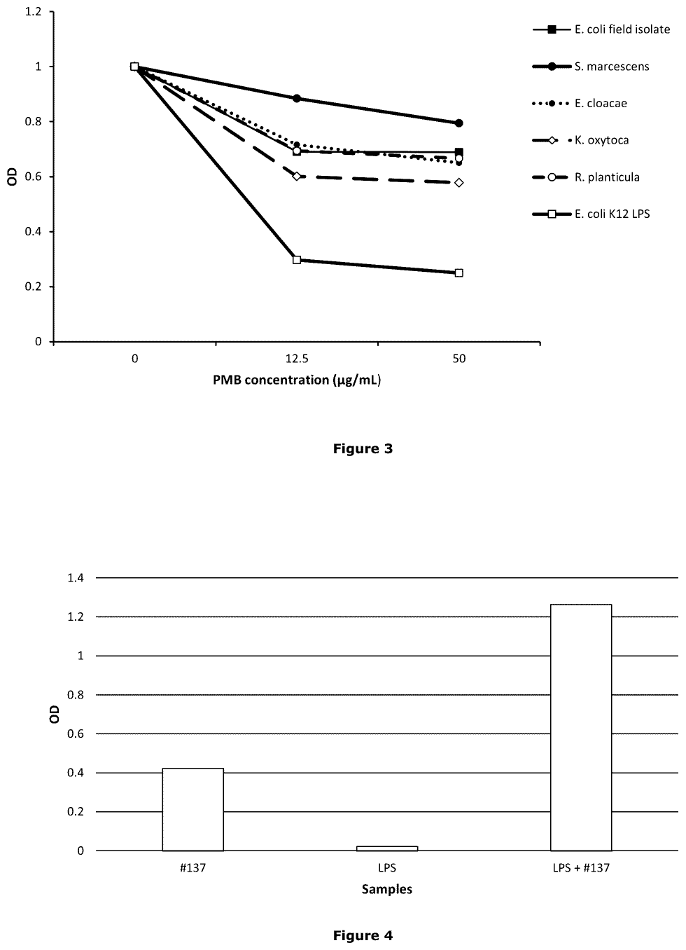

Specific immobilisation by PMB of LPS from a range of different gram-negative bacteria was also demonstrated by pre-incubation with PMB. Sample pre-treatment with PMB effectively competed with LPS binding to immobilised PMB, providing further evidence for specific binding across LPS from different bacterial species. Indeed, competition for binding to PMB was observed in a dose-dependent manner for LPS from an E. coli field isolate, Serratia marcescens, Enterobacter cloacae, Klebsiella oxytoca, Raoultella planticula, and E. coli K12 (see FIG. 3).

### Example 2: Assay Development for the Detection of LPS in Sub-Acute (Mild) Cases of Mastitis

This example presents experiments directed to the development of assay methods suitable for the detection of early or sub-acute disease states, or of less developed infection.

Testing of milk samples from sub-acute cases of E. coli mastitis established that endogenous anti-LPS antibodies may not necessarily be present in all cases. Experiments were conducted to establish if external polyclonal antibodies could be used to generate in vitro an LPS/antibody complex that could then be detected by ELISA.

The source of antibodies was an E. coli-positive milk sample (sample #137) that was found to contain LPS. This milk sample was used to provide the antibodies for other test samples that were found to be devoid of specific antibodies.

The added antibodies were able to bind to LPS in each test sample, which then enabled detection in the ELISA test established above. This approach was further exemplified on LPS present in culture supernatant derived from an E. coli reference ATCC strain (FIG. 4) grown in a milk-based liquid culture.

This approach failed to detect LPS in a Pasteurella multocida sample. Without wishing to be bound by any theory, the applicants interpret this to indicate that the antibody repertoire present in sample #137 is not broad enough to bind to LPS from a divergent gram-negative pathogen.

### Example 3

This example presents experiments directed to the development of assay methods suitable for the detection of a broad range of gram-negative bacteria.

A source of anti-LPS antibodies that contains most if not the entire antibody repertoire against LPS from a wide range of gram-negative bacteria was required. Pooled bovine colostrum was explored as sources of an antibody repertoire broad enough for the detection of LPS from commercially important gram-negative mastitis pathogens.

Antibody preparations from two different sources of pooled colostrum, one originating in the US, the other of New Zealand origin, were provided. Assays were performed according to the methods described in Examples 1 and 2. Samples of differing concentrations of LPS from LSCC #12, E. coli, E. coli #1833, and Serratia maracescens #1831 were analysed.

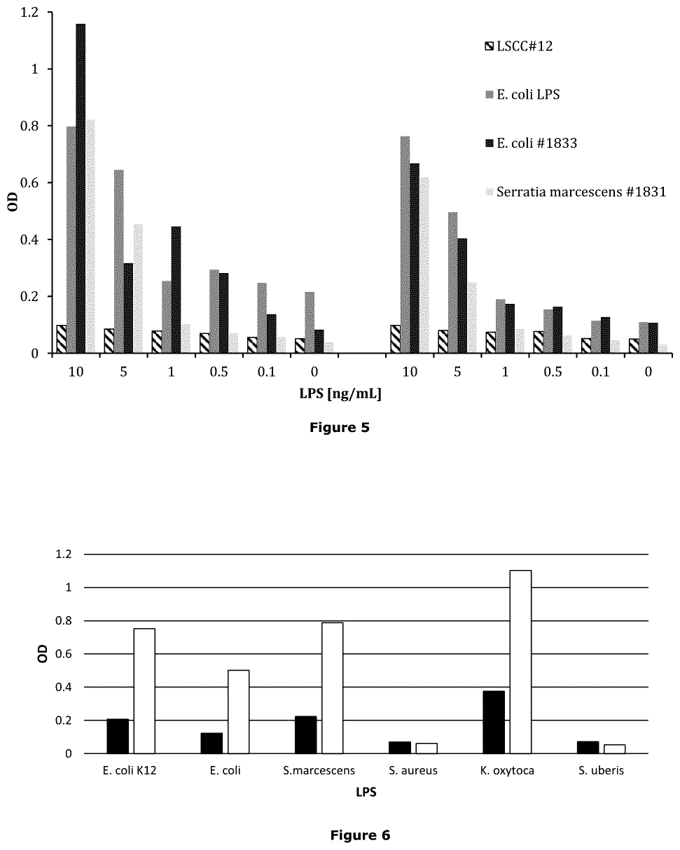

Surprisingly, as shown in FIG. 5 the antibody repertoire differed substantially between the two colostrum pools with indication that the US-sourced pool (FIG. 5, left panel) produces higher sensitivity as well as a better specificity than the NZ-sourced product (FIG. 5, right panel).

### Example 4

This example presents further experiments directed to the development of assay methods suitable for the detection of a broad range of gram-negative bacteria.

Pooled bovine serum was explored as sources of an antibody repertoire broad enough for the detection of LPS from important gram-negative mastitis pathogens.

As shown in FIG. 6 herein, pooled bovine serum does indeed contain antibodies to LPS from a range of gram-negative bacteria. Matched treatment preparations of S. aureus and S. uberis served as negative controls. Notably, the quantitative representation (that is, the amount of LPS-binding activity observed) was substantially lower in serum (FIG. 6, black bars) than in colostrum (FIG. 6, white bars).

### Example 5: Development of a Pan-LPS ELISA with Improved Sensitivity

This example presents further experiments directed to the development of assay methods having improved sensitivity that are suitable for the detection of a broad range of gram-negative bacteria.

The efficacy of the lectin Concanavalin A (ConA) in increasing the sensitivity of LPS detection was assessed.

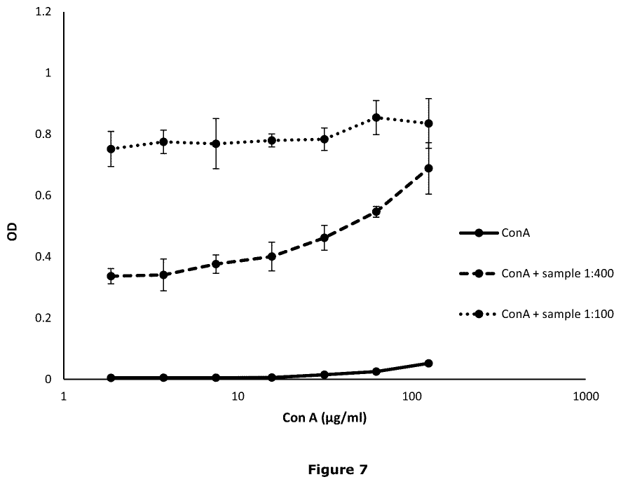

Surprisingly, the addition of ConA to LPS-containing sample at ratios of 1:100 (FIG. 7, dotted line) and of 1:400 (FIG. 7, dashed line) prior to addition of the remaining assay reagents markedly increased the sensitivity of LPS detection. No detection was observed with ConA alone as a negative control (FIG. 7, solid line).

### Example 6: LPS ELISA with Improved Sensitivity

This example presents further experiments exploring the interactions of LPS, ConA, and the other assay reagents to further the development of sensitive assay methods suitable for the detection of LPS analytes.

Experiments were performed to investigate the effect of various agents that were predicted to have potential blocking activity and revealed a complicated interaction between LPS and the various reagents used in the test and with ConA.

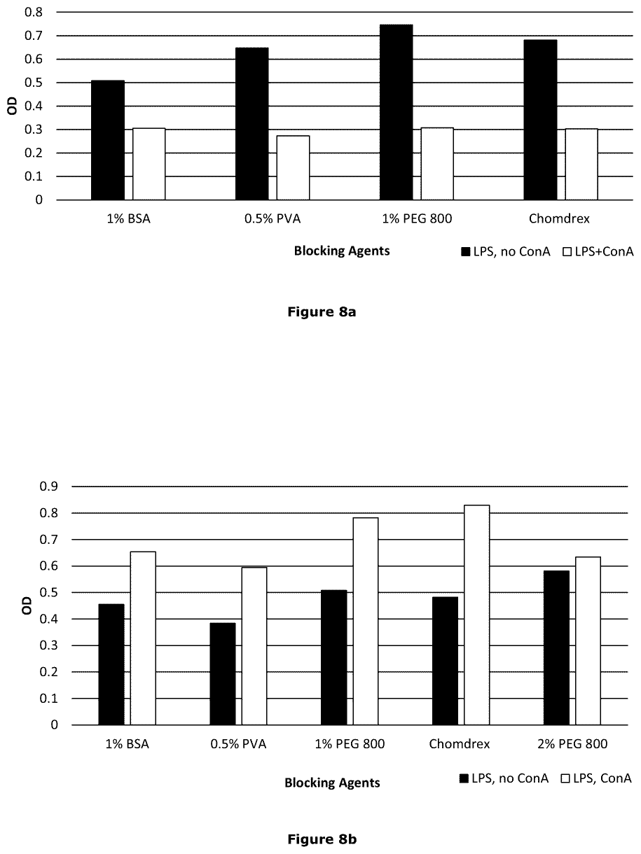

As shown in FIG. 8A, the detection of LPS isolated from bacterial cultures was substantially inhibited by co-incubation with ConA when various potential blocking substances (1% BSA, 0.5% PVA, 1% PEG800, and Chromodrex) were included in separate assays.

In contrast, LPS was readily and sensitively detected when LPS was first captured by immobilized PMB, and the immobilised LPS/PMB complex was then treated with ConA (see FIG. 8b). This effect was observed even in the presence of the various potential blocking agents tested above.

Without wishing to be bound by any theory, the inventors interpret these experiments as indicating that ConA has a novel and surprising beneficial effect on the sensitivity of LPS detection.

### Example 7: Disaggregation of LPS by Triton X-100

This example presents further experiments seeking to understand the interactions of LPS, ConA, and the other assay reagents to further the development of sensitive assay methods suitable for the detection of LPS analytes.

Here, the effect of introducing other assay components, in this case non-ionic detergents, on the sensitivity of LPS detection was explored.

The effect of Triton X-100 and Tween 20 at concentrations from 0.5% to 0.0031% on the sensitivity of LPS detection was assessed. Samples of LPS isolated from six different gram-negative pathogens were used.

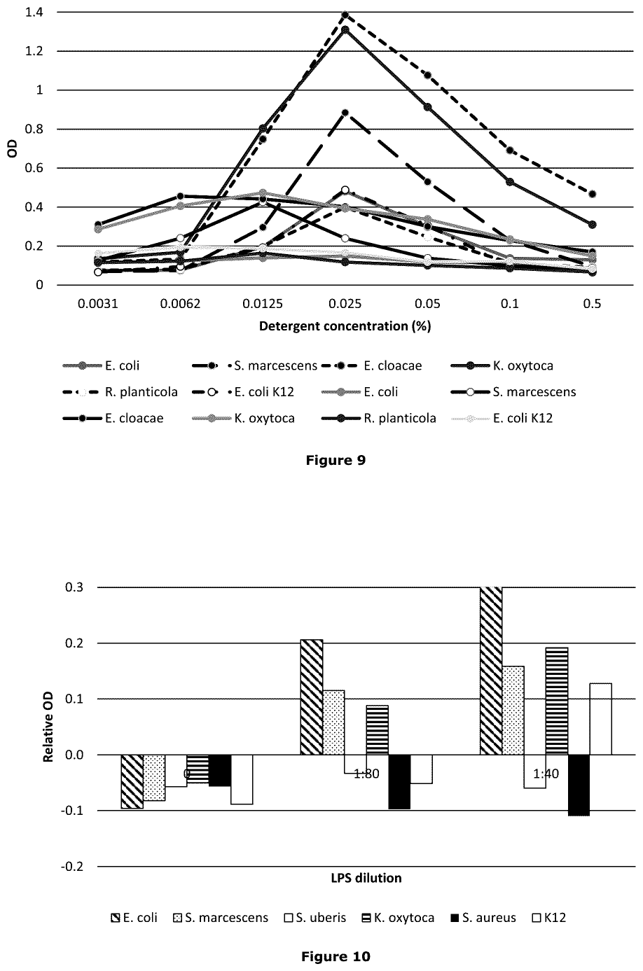

As shown in FIG. 9, pre-treatment of samples with Triton X-100 (solid lines) enabled sensitive LPS detection. In contrast, pre-treatment of samples with Tween 20 (dashed lines) either markedly decreased or prevented detection of LPS.

The highest level of increase in sensitivity was found within a narrow range of Triton X-100 concentrations of 0.0125% to 0.1%. Surprisingly, a clear peak was seen at 0.025% for all LPS preparations although the absolute values were different (see FIG. 9).

Without wishing to be bound by any theory, the inventors hypothesise that sample treatment with Triton X-100 disaggregates LPS by disrupting large LPS micelles and reducing micellar size towards one that is optimal for detection.

### Example 8: Optimal Blocking of ELISA Plates

This example presents further experiments exploring the role of LPS, ConA, and the other assay reagents and their interactions on the sensitivity of assay methods suitable for the detection of LPS analytes.

Here, the effect of introducing other assay components, in this case reagents suspected of functioning as blocking reagents, on the sensitivity of LPS detection was explored.

A range of potential blocking reagents, including BSA, casein, and polyethylene glycols (including PEG-PVAs) in various concentrations, was tested.

Surprisingly, polyethylene glycol 20000 (PEG20) at a specific concentration of 0.05% was found to be the most suitable reagent for improving assay performance. The results showed PEG20 reduced non-specific binding, and improved LPS capture.

Experiments were performed to maximise the capture of LPS from different bacterial samples and at the same time eliminate non-specific interference using a mixture of PMB and PEG20.

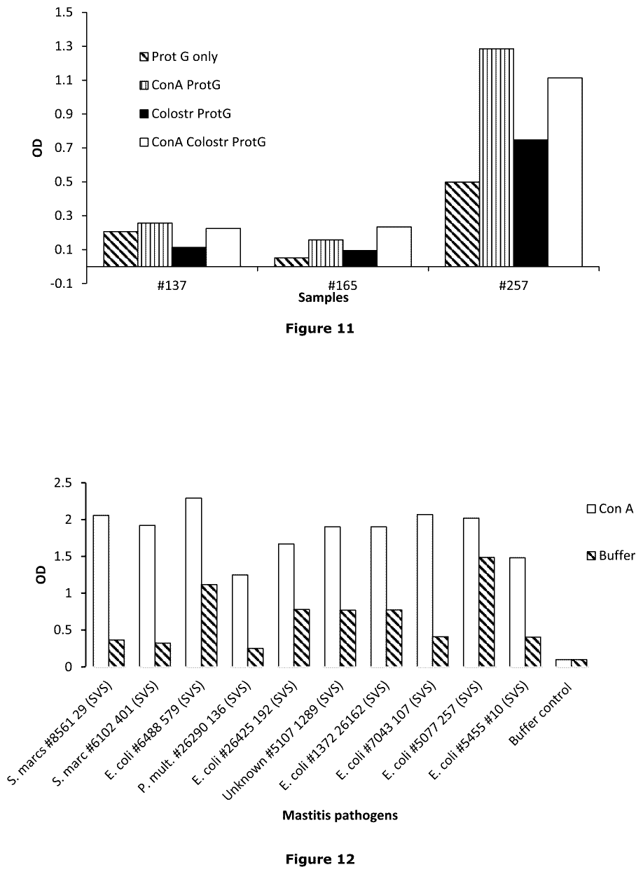

FIG. 10 shows the relative differences in LPS detection observed when microtitre plates were coated with the PMB+PEG20 mixture compared to the sequential coating with PMB followed by blocking with PEG20.

As can be seen, when no LPS was present (LPS dilution “0”, left hand side), coating with the PMB+PEG20 mixture resulted in less non-specific binding. When LPS was present (see FIG. 10, LPS dilution “1:80”, middle; and LPS dilution “1:40”, right hand side), the analytical sensitivity of the assay was improved with the majority of samples when plates were coated with the PMB+PEG20 mixture compared to the sequential coating with PMB followed by blocking with PEG20.

Without wishing to be bound by any theory, the inventors hypothesise that coating of plates with PMB in the presence of an agent such as PEG20 facilitates the interaction and/or orientation of PMB towards one that is optimal for LPS binding.

### Example 9: Mechanism of ConA in Increasing Test Sensitivity

This example presents further experiments exploring the use of ConA to enable sensitive assay methods suitable for the detection of LPS analytes.

Here, the ability of ConA to enable the detection of LPS without a requirement for the addition of exogeneous antibodies was explored.

ConA was found to increase the sensitivity of LPS detection in ELISAs of acute mastitis samples (E. coli mastitis cases #137, #165, #257), even in the absence of external antibodies. In these assays, the presence of LPS-antibody complexes was detected using labelled Protein G.

As shown in FIG. 11, and consistent with the experiments described above, the addition of ConA improved LPS detection compared to assays lacking ConA (compare “ProtG only” with “ConA ProtG”, and “Colostr ProtG” with “ConA Colostr ProtG”). Notably, the addition of colostrum antibodies did not significantly improve the sensitivity of detection of LPS present in these acute E. coli mastitis cases—compare “ConA ProtG” with “ConA Colostr ProtG”.

Samples from mastitis cases of various severity were then tested to determine if the addition of colostrum antibodies is beneficial in a range of cases. As shown in FIG. 12, these experiments established that LPS from mastitis cases of differing severity and resulting from infection by various pathogens was efficiently detected by treatment with ConA followed by labelling with colostrum antibodies. In each case, ConA improved LPS detection. Importantly, LPS from a wide range of relevant gram-negative mastitis pathogens (e.g. E. coli, S. marcescens, Pasteurella multocida) was able to be detected. This demonstrates that antibodies in colostrum pools of specific origins have an antibody repertoire that is suitable for the detection of LPS from most gram-negative mastitis pathogens, particularly when assay sensitivity is improved with ConA. The detection of P. multocida LPS, which lacks O-antigens, is indicative of detection of conserved parts of the molecule.

Without wishing to be bound by any theory, the inventors interpret this to support their view that the methods disclosed herein can be effective in detecting LPS with O-antigens that are not recognized by colostrum (or other) antibodies, thereby broadening the range of potential target gram-negative species able to be detected.

### Example 10: Assessing the Activity of Other Lectins

This example presents further experiments exploring the use of ConA and of other lectins to enable sensitive assay methods suitable for the detection of LPS analytes.

Here, the ability of representative lectins soybean agglutinin (SBA, with affinity for galactose (Gal) and N-acetyl galactosamine) and Crotalaria juncea lectin (CJL, isolated from Sunn Hemp with specificity toward Gal over GaINA) to enhance the detection of LPS was explored.

As shown in FIG. 13, addition of CJL did not improve the detection of LPS in the tested milk samples, but SBA slightly improved the detection of LPS from S. marcescens and in one of the E. coli cases. In all but one case, addition of ConA resulted in markedly improved LPS detection.

These data show that the lectin ConA is highly beneficial in increasing the sensitivity of LPS detection across a range of pathogenic bacteria. In contrast, treatment with SBA had only a minor beneficial effect, while CJL treatment was ineffective.

### Example 11: Assay Conditions and Mechanism of ConA in Increasing Test Sensitivity

This example presents further experiments exploring the use of ConA to enable sensitive assay methods suitable for the detection of LPS analytes.

Here, the assay conditions in which the ability of ConA to best enhance the detection of LPS were explored.

It was established that when ConA was used at a slightly alkaline pH, no beneficial effect on the sensitivity of LPS detection was observed (data not shown). In contrast, ConA storage and application at an acidic pH (here, pH of 5.4) followed by an increase to pH 7.2 effectively aided LPS detection.

It has been reported that ConA can reversibly transform from a dimeric form to a tetrameric configuration when the pH is changed (Auer, H. E. & Schilz, T. pH-dependent changes in properties of concanavalin A in the acid pH range. Int. J. Pept. Protein Res 24, 462-471 (1984)). Without wishing to be bound by any theory, the inventors interpret these results to indicate that the addition of ConA followed by a pH dependent transformation to the tetrameric form promotes the formation of stable LPS epitopes to which antibodies can bind with high affinity.

Notably, the beneficial effect of ConA addition and of the dimer/tetramer transformation were observed when LPS/antibody complexes were already present in the biological sample, such as in the case of acute E. coli mastitis, and when the addition of exogeneous antibodies was required for improved LPS detection.

### Example 12: Exploring the Specificity of the LPS Assay

This example presents further experiments directed to the development of assay methods having improved sensitivity that are suitable for the detection of a broad range of gram-negative bacteria.

Here, the specificity of the assay was assessed by testing LPS prepared from a range of gram-negative mastitis pathogens and from human sepsis pathogens. A source of bovine colostrum antibodies was used.

LPS samples from relevant mastitis pathogens (E. coli, Klebsiella oxytoca, S. marcescens, Raoultella planticula, E. cloacae) and from E. coli K12 were used for specificity testing. Three gram-positive mastitis isolates (S. aureus, Coagulase negative Staphylococci (CNS), and S. uberis) served as negative controls for the assay. A commercially sourced LPS from an O-antigen deficient E. coli strain (K12) was included to demonstrate the antibody repertoire of colostrum includes antibodies that bind to conserved core antigens to or lipid A antigens.

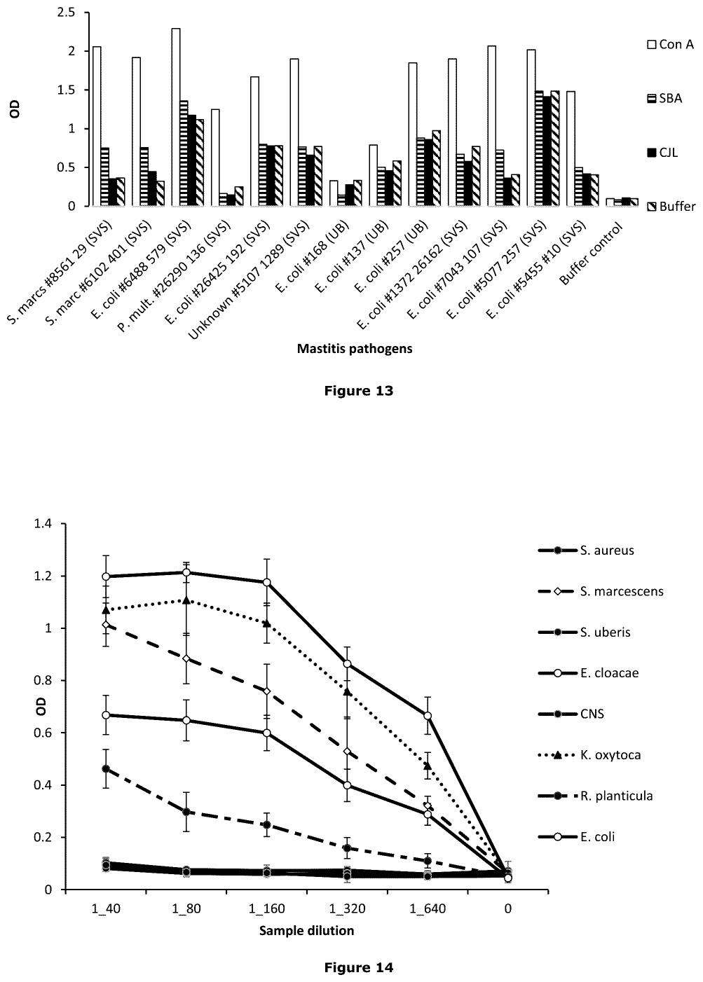

As shown in FIG. 14, the antibody repertoire in bovine colostrum was sufficiently large so as to enable detection of LPS from each of gram-negative mastitis samples representing a large range of mastitis pathogens (FIG. 14). No cross-reactivity was observed with the three gram-positive species, although antibodies to these mastitis pathogens were found to be present (data not shown).

These experiments clearly demonstrated that the assay has a high analytical specificity for gram-negative mastitis pathogens, and is capable of detecting infection from a broad range of pathogenic bacteria.

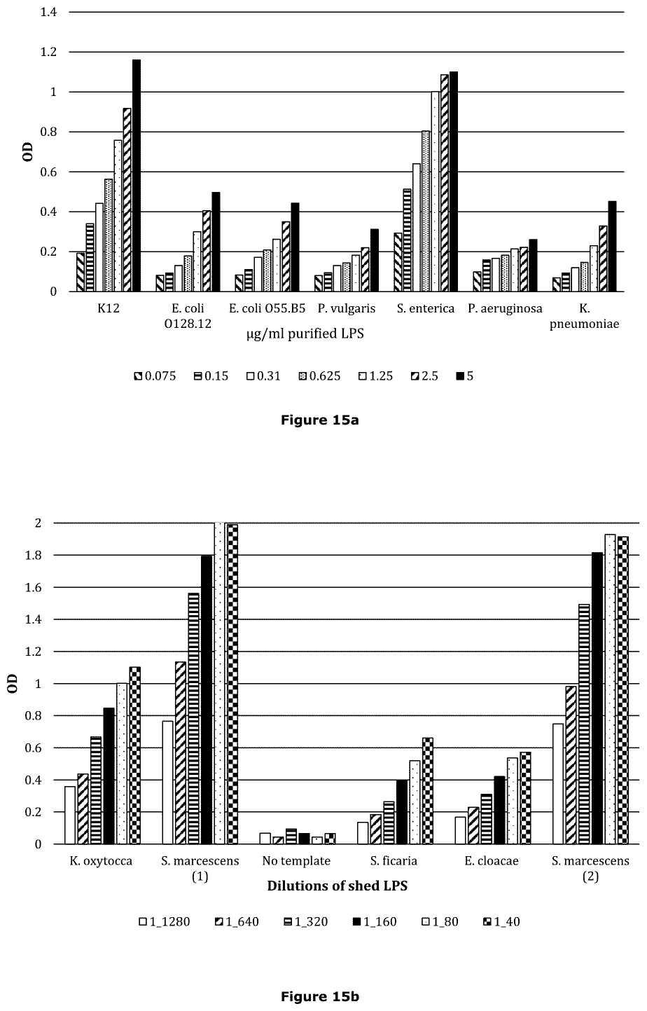

The data presented in FIG. 14 showing the detection of LPS from facultative human pathogens (E. coli, Serratia marcescens, E. cloacae) indicated that the assay was able to detect LPS from these pathogens. The data presented in FIGS. 15a and 15b, showing specificity of the assay for a wide range of LPS from gram-negative human pathogens including LPS from E. coli O128.12, E. coli O55.B5, Proteus vulgaris, Salmonella enterica, Pseudomonas aeruginosa, Klebsiella pneumoniae, K. oxytoca, S. ficaria, S. marcescens and E. cloacae.

This clearly supports the utility of the assay in the detection of LPS from gram-negative pathogens responsible for disease causing infections including sepsis in humans and other mammals.

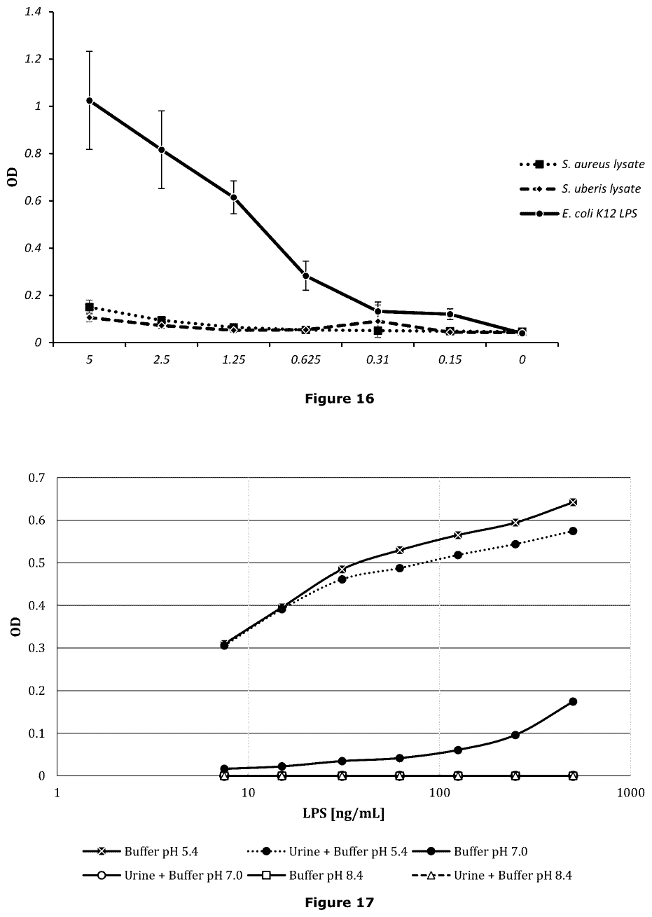

The data presented in FIG. 16, showing the ability of the assay to detect E. coli K12 LPS, demonstrates that a colostrum pool contains antibodies that are specific for the conserved parts of the LPS molecule. This is evidenced by the fact that in the absence of highly variable O-antigens, binding to the relatively conserved core antigens and/or highly conserved lipid A antigens was seen (FIG. 16). Again, the specificity for antigens from gram-negative bacteria was clearly evidenced, as antigens from the gram positive species S. aureus and S. uberis do not react in the assay.

Without wishing to be bound by any theory, this finding supports the view that even in the absence of species-specific or strain-specific antibodies in the colostrum pool, the sensitive detection of LPS from a wide range of gram-negative human pathogens is possible due to the presence of antibodies binding to both species- or strain-specific LPS components and of antibodies recognizing conserved parts of the LPS molecule.

### Example 13: Assay for LPS Detection in Urine Samples

This example presents experiments directed to the development of assay methods for the detection of LPS in urine samples. Here, a direct ELISA method utilises anti-LPS antibodies present in colostrum to detect LPS.

### Methods

### LPS Spiked Urine Samples.

Bovine, ovine and canine (collected into endotoxin-free sterile containers) were spiked with (i) naïve LPS shed from Gram-negative bacteria cultures or (ii) commercially purchased (Sigma) purified LPS standards. The LPS concentration range used was 0.15-1000 ng/mL. This concentration range usefully covers the range of endotoxins typically encountered in human and animal clinical settings.

The samples were mixed thoroughly and then incubated at room temperature (RT, 20° C.) for 30 mins. Samples were then either measured by ELISA or frozen −20° C.

### LPS Urine Sample Preparation for ELISA.

Samples were diluted 1:10 in 0.1M sodium citrate buffer, pH 5.4 containing 0.025% Triton X-100, mixed by vortexing and then incubated at RT for 30 mins. A number of other buffer/detergent/pH combinations were examined but were considered less optimal.

### ELISA Method

Briefly, 96-well microplates (medium binding affinity) coated overnight at RT with 50 μg/mL polymyxin B diluted in 0.25% PEG20/phosphate buffered saline (PBS) were washed twice with PBS. LPS spiked urine dilutions (100 uL) were added to wells and incubated for 1 hr at 37° C. The plates were then washed 3× with PB-Tween 20 (PBST) and then blocked with a 4% BSA/PBS solution (120 uL)/well) for 30 mins at RT and then washed 1× with PBST. Next, LPS captured by polymyxin B was detected using colostrum (German pulvar concentrated in bovine IgG & IgA antibodies, diluted 1:2000 in PBS) was added to wells (100 uL) and incubated at 37° C. for 30 mins. The plates were then washed 4 times in PBST followed by an incubation with a 100 uL protein G-HRP conjugate (Assure Quality, diluted 1:100 in PBST) and a final plate wash comprising 5 washes with PBST. Enzyme activity (HRP) was measured using TMB colorophore and its substrate (100 uL) for 10 mins at RT and then stopped using 0.1M sulphuric acid (50 uL). The absorbance of the final solutions was measured at wavelengths 450 nm and 650 nm on a plate-reader. Data was calculated as difference between 450 nm-650 nm and currently expressed as a change from baseline absorbance using urine alone samples.

### Results

### Identification of a Suitable LPS Extraction Method for Urine

Three different sample preparation methods (acidic, neutral and alkaline pH) were evaluated for suitability of LPS detection in urine. Only 0.1M Na⁺ citrate/0.025% Triton X-100, pH 5.4 but not 0.1M Na⁺PO4⁻/0.05% Triton X-114, pH 7.0 or 0.1Tris/HCl, 1 mM EDTA, pH 8.4 was found to be suitable for sample preparation (FIG. 17) in an ELISA that utilised PMB capture, colostrum antibody labelling and Protein G based detection.

FIG. 17 presents the results of this testing of various sample extraction buffers. As can be seen, re-isolation and detection of LPS from spiked urine samples was only possible with the use of an acid (citrate buffer pH 5.4), but not with a neutral (phosphate buffer pH 7.0) or alkaline (Tris buffer pH 8.4) sample diluent.

The results depicted in FIG. 17 also showed that this pH 5.4 sample dilution buffer while effective was suboptimal for some clinical applications due to a relatively high background and a lack of linearity at higher LPS concentrations.

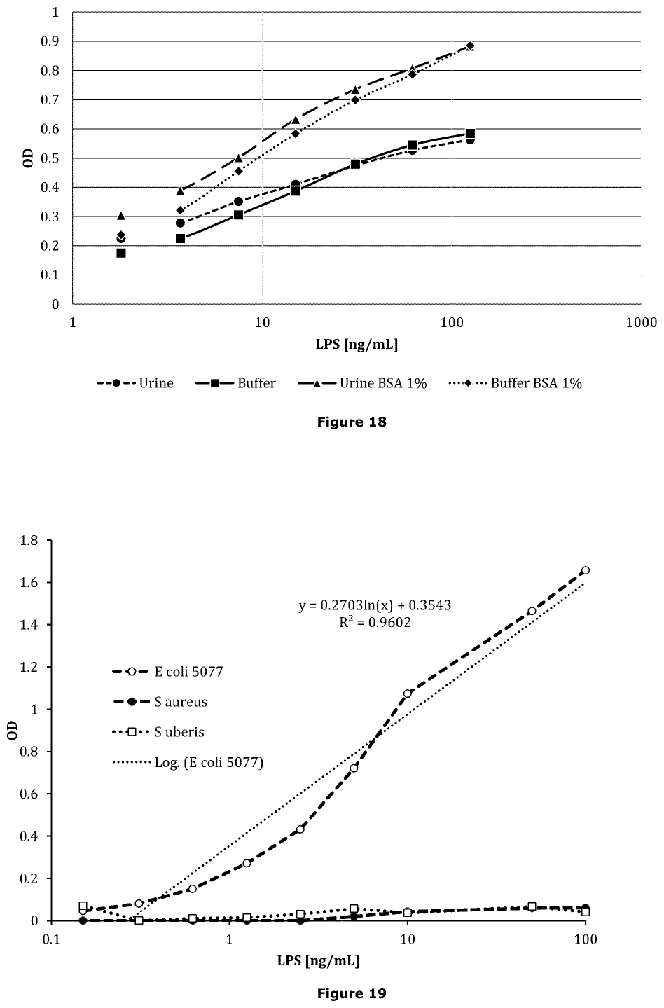

An improvement of the ELISA performance was achieved by the addition of BSA after sample addition. The addition of BSA not only reduced the ELISA background (which may be consistent with BSA acting as a blocking reagent to prevent non-specific binding of LPS or LPS/antibody complexes to the ELISA plate), but surprisingly also improved the linearity of the assay over a wider range of LPS concentrations, as shown in FIG. 18). Without wishing to be bound by any theory, it appears that the addition of BSA facilitates an improved ability for antibodies to bind to LPS.

Further optimisation of the BSA concentration then revealed that a 4% BSA solution was found to be better than a 1% BSA (data not shown).

### Detection of LPS in Spiked Urine Samples

Bovine urine was spiked with E. coli 5077 LPS or with extracts from gram positive bacteria (S. aureus or S. uberis). As can be readily seen in FIG. 19, specificity of this assay for gram-negative LPS was demonstrated by detection of LPS from E. coli 5077 over a wide range of clinically relevant concentrations. Notably, samples comprising preparations from the Gram-positive bacteria S. aureus or S. uberis showed no appreciable binding.

These data shown that LPS from Gram-negative species that can cause urinary tract infections could be detected in bovine urine with high specificity in an assay that is highly specific for Gram-negative bacteria.

### Example 14: Assay for LPS Detection in Urine Samples

This example presents experiments further exploring assay methods for the detection of LPS in urine samples from different animal species.

### Methods

Assays were performed on urine samples obtained from bovine, ovine, and canine subjects using the methodology including the addition of 4% BSA as set out in Example 13 above.

### Results

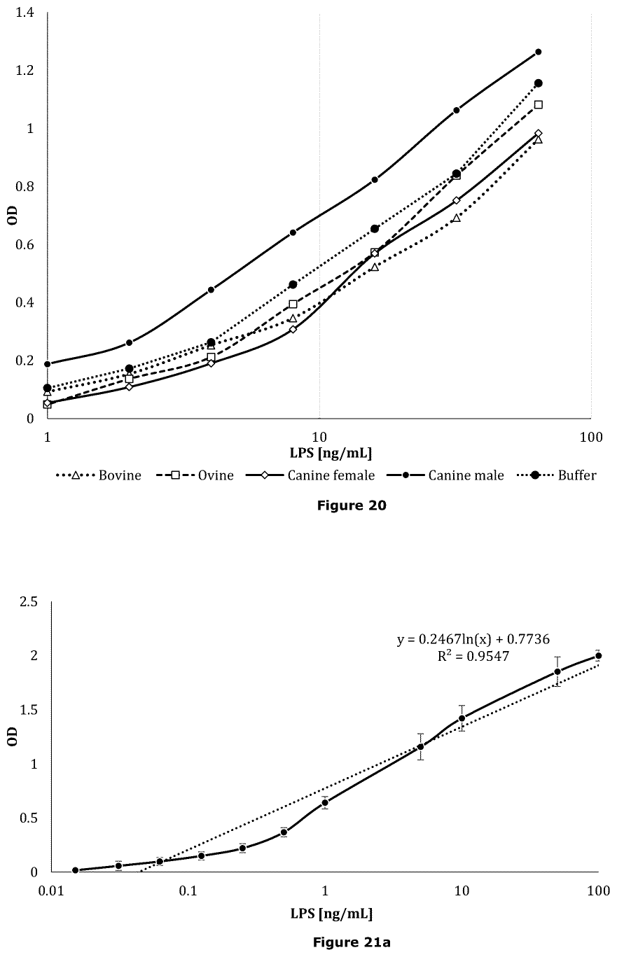

Urine from bovine, ovine or canine (female and male) subjects was spiked with various amounts of E. coli 5077 LPS. As can be seen in FIG. 20, LPS was detected with a high level of sensitivity in all samples over a wide range of concentrations, and in the LPS-containing buffer positive control. The data presented in FIG. 20 shows this assay had an LPS detection limit below 1 ng/ml.

### Example 15: Assay for LPS Detection in Urine Samples

This example presents experiments further exploring assay methods for the detection of LPS from different pathogenic bacteria in canine urine samples.

### Methods

LPS from the pathogenic bacteria S. marcescens, K. oxytoca, and E. coli 5077, was added in varying amounts to canine urine. Assays were performed on urine samples obtained from canine subjects using the methodology including the addition of 4% BSA as set out in Example 13 above.

### Results

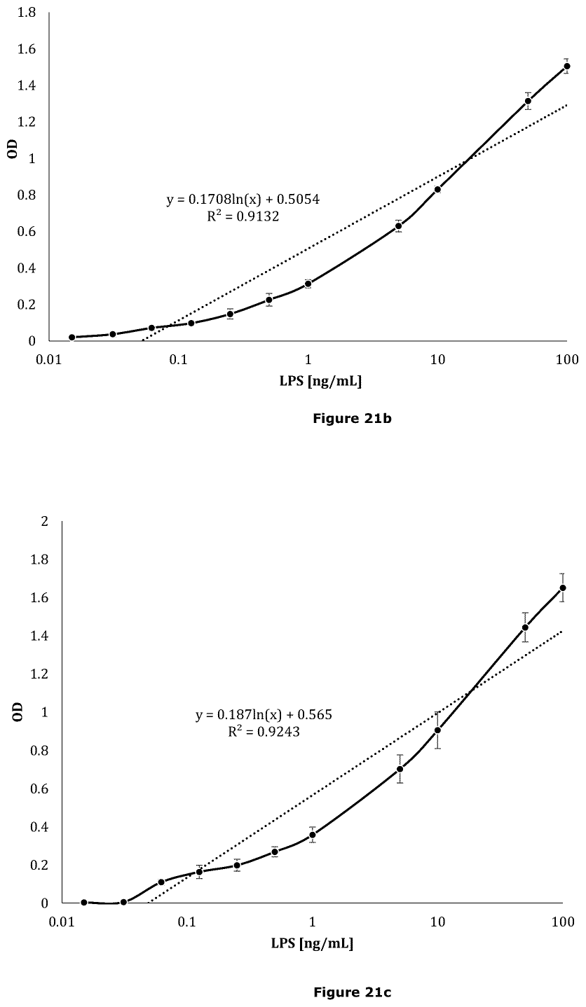

As can be seen in FIGS. 21a, 21b, and 21c, LPS from E. coli 5077 (FIG. 21a), S. marcescens (FIG. 21b) and K. oxytoca (FIG. 21c) were readily detectable in canine urine samples.

Notably, the lower limit of detection for LPS from each species was below 100 pg/mL, while the upper limit of detection was above 100 ng/mL.

Examples 13-15 above show that LPS from Gram-negative bacterial species that can cause urinary tract infections could be detected in bovine urine with high specificity (Example 13) and in urine samples from three different animal species (Example 14). Example 15 establishes that exogeneous LPS from three different bacterial species can be detected in canine urine.

### Example 16: Detection of Acute Mastitis

This example presents experiments further exploring assay methods for the detection of LPS/IgG complexes in milk samples from bovine with mastitis as a representative model of infection with coliform bacteria.

### Methods

Assays were performed using the method described in Example 13 on milk samples obtained from bovine subjects, except for the following alterations. The samples were diluted in a dilution buffer comprising 0.1Tris/HCl, 1 mM EDTA, pH 8.4. Samples were incubated for 30 mins at 21° C. with PMB (50 ug/mL) diluted in 0.25% PEG20/PBS, pH 7.4. The detection step utilised the German colostrum enriched antibody preparation (as described above, 1:2000 dilution) followed by AQ-protein-G (1:80 dilution). Enzyme activity (using 100 uL per ELISA) was measured with AQ-TMB and substrate buffer.

### Results

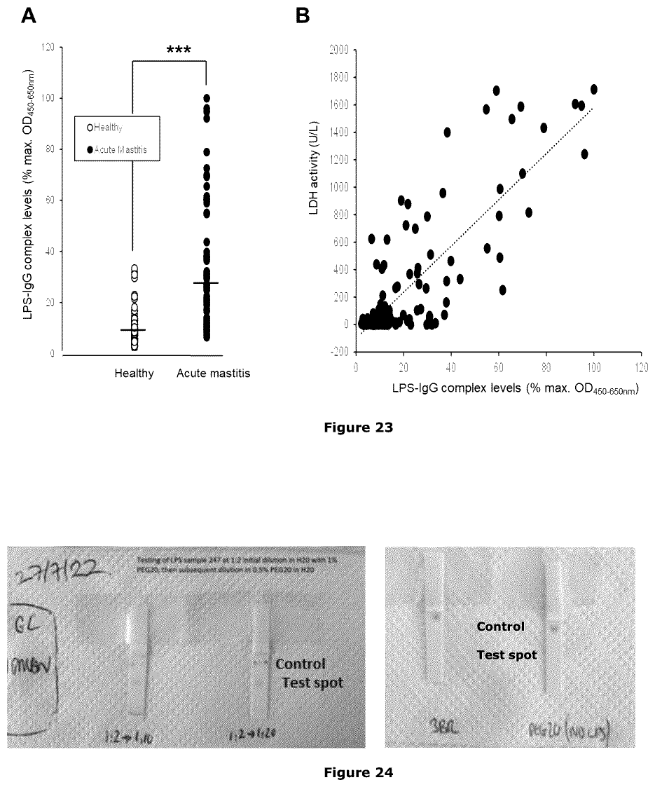

As shown in FIG. 23, LPS/IgG complexes were found in samples from acute coliform mastitis, but not in healthy milk samples (FIG. 23A). The presence of LPS/IgG complexes correlated well with inflammation as measured by LDH activity (FIG. 23B).

These data show that the detection of existing LPS/IgG complexes in milk from acute mastitis cases is efficaciously achieved using the ELISA methods presented here. Furthermore, without wishing to be bound by any theory, applicants infer that the presence of LPS/IgG complexes in mastitis milk is representative of infection with coliform bacteria.

### Example 17: Lateral Flow

This example presents experiments exploring a lateral flow assay format for the detection of LPS/IgG complexes in milk samples from bovine with mastitis as a representative model of infection with coliform bacteria.

### Methods

Broadly, LPS/IgG complexes present in mastitis milk were captured by PMB or alternatively with a PMB nanopeptide. The immobilised complex was then visualised by either a Protein G 40 nm nanogold conjugate, or with a Protein A/G 150 nm nanoshell (NS) conjugate.

Samples were extracted using a two-step extraction procedure and are performed on the raw milk sample. The initial dilution was performed at a 1:2 dilution in 1% PEG20 in RO water followed by a 1:5 dilution in 0.5% PEG20 in RO water, or alternatively in a single dilution step of 1:6.5. This extraction procedure enabled flow of the sample in the nitrocellulose membrane without any additional treatment. Omitting this sample extraction step reduced or entirely impeded sample flow and prevented the interaction between the immobilised PMB and the LPS/IgG complex in the sample.

The conjugate concentrates (Protein G/40 nm nanogold or Protein A/G 150 nm nanoshells) required dilution in 5-10% Tween 80 for optimal performance. A 1% Tween 80 solution did not enable flow of conjugate.

### Results

A positive signal was obtained with samples verified for the presence of an LPS/IgG complex but not with samples that lack LPS/IgG. An example of a weak LPS positive milk sample is given in FIG. 24, left panel, where the test spot can readily be observed. In contrast, mastitis negative samples and a non-LPS buffer control gave no test signal (FIG. 24, right panel).

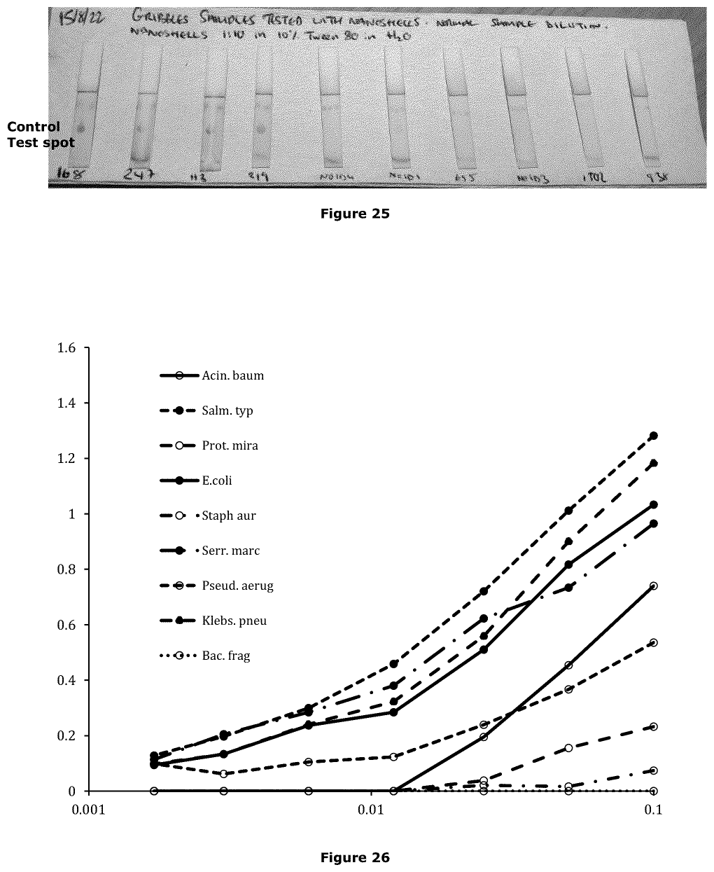

The detection of existing LPS/IgG complexes in a LF format assay was also demonstrated by using a Protein A/G/150 nm nanoshell (NS) conjugate. A set of acute mastitis samples was tested using a two-step sample extraction protocol followed by NS dilution in 10% Tween 80. This test revealed that an LPS/IgG complex can be reliable detected (Table 1 and FIG. 25). As shown in Table 1, the lateral flow test results correlated well with ELISA results.

TABLE 1
Lateral flow results in comparison to LPS ELISA and culture

| | | LPS | | |
|---|---|---|---|---|
| Sample ID | LF | ELISA | Culture | Comment |
| 168 | Highly Pos | Pos | E. coli | High PC |
| 247 | Pos | Pos | E. coli | Low PC |
| 113 | Highly Pos | Pos | S. dysgalactia | Mixed infection? |
| 219 | Highly Pos | Pos | S. uberis | Mixed infection? |
| No ID4 | Neg | Neg | S. aureus | S. aureus |
| No ID1 | Pos | Pos | Serratia | |
| 655 | Neg | Neg | Corynebacterium | |
| No ID3 | Neg | Neg | S. uberis | |
| 1502 | Neg | Neg | S. aureus | |
| 938 | Neg | Neg | Pseudomonas | Contamination? |

### Example 18. Detection of Human Sepsis-Causing Pathogens

This example presents experiments exploring methods for the detection of LPS/IgG complexes in human clinical samples from humans with sepsis.

### Methods

Human sepsis pathogens were clinical isolates from patients in Palmerston North, New Zealand. Characterisation of pathogens and LPS extraction was performed by Medlab Central, Palmerston North. Testing by ELISA used PMB-based capture, ConA based multimerization, labelling with colostrum antibodies and detection via Protein G-HRP as herein described.

### Results

As can be seen in FIG. 26, the method allowed detection of LPS from Acinetobacter baumannii, Salmonella typhimurium, Proteus mirabilis, Escherichia coli, Serratia marcescencs, Pseudomonas aeruginosa and Klebsiella pneumoniae but not for LPS from Bacillus fragilis. E. coli LPS isolated from an animal mastitis case and K12 LPS served as positive controls. No reaction was observed with membrane fragments from a sepsis isolate of Staphylococcus aureus. This data therefore shows reactivity for a wide range of Gram-negative sepsis pathogens apart from B. fragilis (FIG. 26), and excellent discrimination between sepsis caused by Gram-negative bacteria and sepsis caused by Gram-positive bacteria.

### Example 19. Detection of LPS in Plasma Samples

This example presents experiments exploring methods for the detection of LPS/IgG complexes in plasma samples prepared from bovine and ovine blood.

### Methods

The method described in this example focused on the detection of LPS in plasma. Initial experiments showed that components of plasma usually prevent the detection of LPS or make it unreliable.

Bovine and ovine blood was used as a model for human blood samples in order to develop methods to enable detection of LPS in plasma using an ELISA format. The method used LPS capture by PMB/PEG20, followed by labelling with colostrum antibodies and detection with Protein G-HRP, as described herein.

### Results

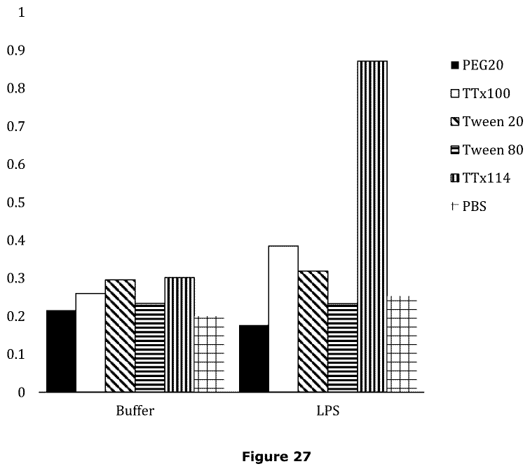

Assays were developed in which the unmasking of LPS in plasma that was spiked with LPS was achieved. Here, the addition of Triton X-114, but not of PEG20, Triton X-100, Tween20, Tween80 or PBS, enabled unmasking of LPS and its detection (FIG. 27).

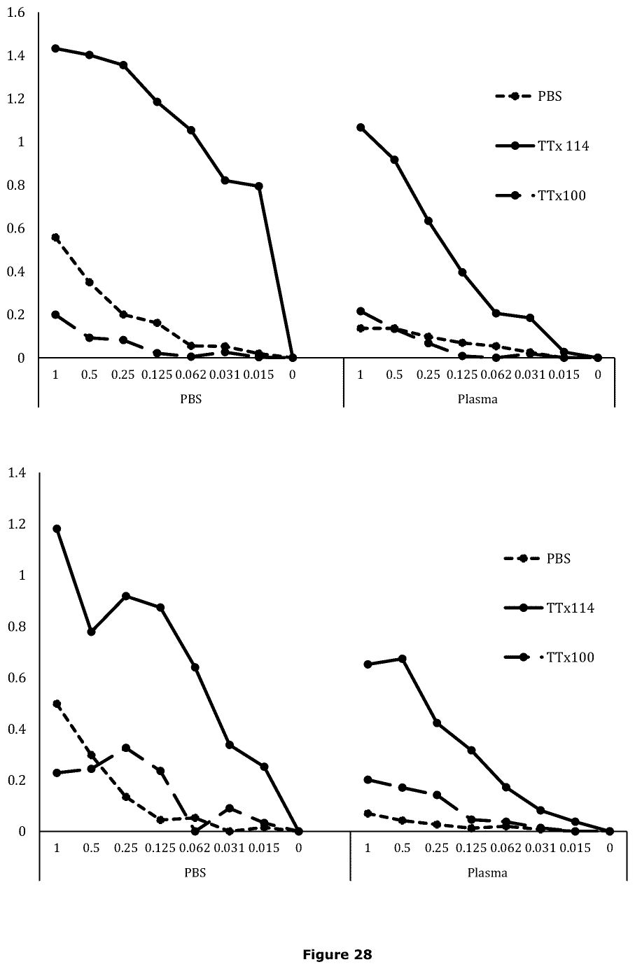

Additional experiments established that LPS from two different pathogens can be detected in spiked plasma, in this case extraction with Triton X-114 being superior to extraction with Triton X-100 or PBS (FIG. 28). Here, sample pre-treatment with Triton X-114 enabled the detection of E. coli LPS (FIG. 28, top) or Serratia marcescens LPS (FIG. 28, bottom) in plasma over a wide range of concentrations.

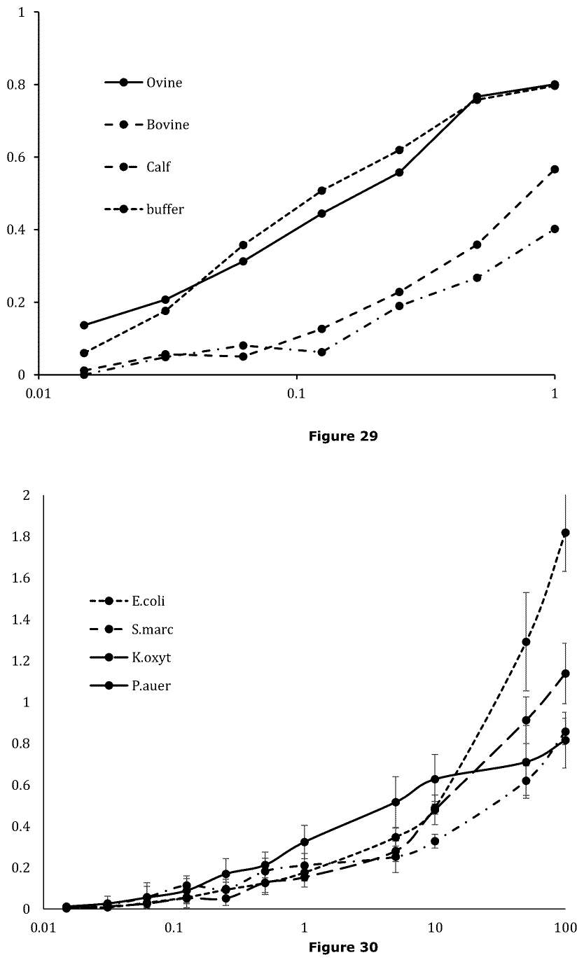

Additionally, experiments established that a PEG concentration of 0.25% in the PMB capture buffer substantially improved background. As can be seen in FIG. 29, the inclusion of 0.25% PEG20 reduced the background observed when detecting LPS in ovine and bovine plasma samples to levels comparable to those observed when the LPS to be detected was present in a simple buffer system.

### Example 20. Detection of LPS in Plasma Samples

This example presents further experiments exploring methods for the detection of LPS in plasma samples prepared from ovine blood.

### Methods

The method described in this example focused on the detection of LPS in plasma. Ovine plasma was spiked with 10× naïve LPS generated using coliforms (Pseudomonas aeruginosa, K. oxytoca, S. marcescens and E. coli) cultured from acute mastitis cow's milk.

Plasma samples were diluted 1:10 in ice cold 1% Triton X-114/0.1 Na⁺ phosphate buffer, pH 7.0. Diluted samples were then vortexed and incubated at RT (20° C.) for 30 mins and voertexed again.

Medium affinity binding microplates (Costar strips) were coated with 50 μg/mL PMB diluted in 0.25% PEG20/PBS, pH 7.4 for 16 hrs at RT. Plates were washed two times with PBS, then 100 μL of sample dilutions were added to wells and incubated for 90 mins at 37° C. Plates were then washed three times with PBST. 120 uL 2% BSA/PBS solution was added to wells and incubated at RT for 30 mins. Then, plates were washed twice with PBST. 100 μL of colostrum (1:200 dil of 10% German colostrum stock in PBS) was added to wells and incubated for 30 mins at 37° C. Plates were then washed four times in PBST. 100 uL protein G-HRP (1:100 dil AQ protein G HRP in PBST) was added to wells and incubated for 35 mins at 37° C. Plates were then washed five times with PBST, and enzyme activity was then measured using AQ-TMB diluted in substrate buffer for 10 mins at RT.

### Results

As can be seen in FIG. 30, highly sensitive detection (down to below 500 pg/mL) of LPS from each of the bacteria P. aeruginosa, K. oxytoca, S. marcescens and E. coli was achieved in this assay.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications and additions may be made without departing from the scope of the invention, for example when present the invention as defined in the indicative claims. Furthermore, where known equivalents exist to specific features, such equivalents are incorporated as if specifically referred in this specification.

1. A method of determining the presence of a lipopolysaccharide analyte in a biological sample, the method comprising the steps of:
(a) a lipopolysaccharide analyte capture step, comprising contacting the sample with at least one capture reagent in the presence of a solid substrate, said capture reagent capable of binding to lipopolysaccharide;
(b) contacting the sample with a multimerizing reagent capable of binding to lipopolysaccharide, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with a binding reagent capable of binding to the complex comprising lipopolysaccharide analyte and multimeric multimerizing reagent;
(e) detecting the presence of the complex so formed;
and wherein the presence of the complex is indicative of the presence of a lipopolysaccharide analyte in the sample.

2. A method of determining the presence of gram-negative bacteria in a sample or in a subject from whom a sample has been obtained, the method comprising
(a) a lipopolysaccharide analyte capture step, comprising contacting the sample with at least one capture reagent in the presence of a solid substrate, said capture reagent capable of binding to lipopolysaccharide;
(b) contacting the sample with a multimerizing reagent capable of binding to lipopolysaccharide, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with binding reagent capable of binding to the complex comprising lipopolysaccharide analyte and multimeric multimerizing reagent;
(e) detecting the presence of the complex so formed;
wherein the presence of the complex is indicative of the presence of gram-negative bacteria in the sample or in the subject from whom the sample was obtained.

3. A method of diagnosing mastitis in a subject or of identifying a subject at increased risk of having or developing mastitis, the method comprising the steps:
(a) a lipopolysaccharide analyte capture step, comprising contacting the sample with at least one capture reagent in the presence of a solid substrate, said capture reagent capable of binding to lipopolysaccharide;
(b) contacting the sample with a multimerizing reagent capable of binding to lipopolysaccharide, wherein when contacted with the sample said multimerizing reagent is capable of further multimerization;
(c) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(d) optionally contacting the sample with binding reagent capable of binding to the complex comprising lipopolysaccharide analyte and multimeric multimerizing reagent;
(e) detecting the presence of the complex so formed;
wherein the presence of the complex indicates that the subject has mastitis or is at increased risk of developing mastitits.

4. A method of determining the presence of a lipopolysaccharide analyte in a biological sample or of determining the presence of gram-negative bacteria in a sample or in a subject from whom a sample has been obtained, the method comprising the steps of:
(a) contacting the sample with at least one capture reagent and at least one multimerizing reagent, said capture reagent capable of binding to lipopolysaccharide or to a complex in which lipopolysaccharide is present, and said multimerizing reagent capable of binding to lipopolysaccharide or a complex in which lipopolysaccharide is present, wherein when contacted with the sample said multimerizing reagent is capable of further multimerization;
(b) maintaining the lipopolysaccharide analyte and the multimerizing reagent in contact for a time and under conditions suitable for multimerization of the multimerizing reagent and the formation of a complex comprising lipopolysaccharide analyte and multimeric multimerizing reagent;
(c) optionally contacting the sample with a binding reagent capable of binding to the complex comprising lipopolysaccharide analyte and multimeric multimerizing reagent;
(d) detecting the presence of the complex so formed;
wherein the presence of the complex is indicative of the presence of a lipopolysaccharide analyte in the sample or is indicative of the presence of gram-negative bacteria in the sample or in the subject from whom the sample has been obtained.

5. A method of determining the presence of a lipopolysaccharide analyte in a biological sample or of determining the presence of gram-negative bacteria in a sample or in a subject from whom a sample has been obtained, the method comprising the steps of:
(a) a lipopolysaccharide analyte capture step, comprising contacting the sample with at least one capture reagent, said capture reagent capable of binding to lipopolysaccharide or to a complex in which lipopolysaccharide is present;
(b) maintaining the lipopolysaccharide analyte and the capture reagent in contact for a time and under conditions suitable for the formation of a complex comprising lipopolysaccharide analyte and capture reagent;
(c) optionally contacting the sample with an albumin, such as bovine serum albumin, or an ovalbumin;
(d) optionally contacting the sample with a binding reagent capable of binding to the complex comprising lipopolysaccharide analyte and capture reagent;
(e) contacting the sample with an immunoglobulin binding protein;
(f) detecting the presence of the complex so formed;
and wherein the presence of the complex is indicative of the presence of a lipopolysaccharide analyte in the sample or is indicative of the presence of gram-negative bacteria in the sample or in the subject from whom the sample has been obtained.

6. The method according to claim 5, wherein the sample is contacted with the at least one capture reagent in the presence of one or more non-ionic detergents.

7. The method according to claim 6, wherein the method comprises the preliminary step of contacting the sample with one or more non-ionic detergents prior to contact with the one or more capture reagents.

8. The method according to any one of claims 1 to 7, wherein the at least one capture reagent is immobilised on the solid support.

9. The method according to any one of the preceding claims wherein the lipopolysaccharide analyte in the sample is derived from the cell membrane of gram-negative bacteria.

10. The method according to any one of the preceding claims wherein the gram-negative bacteria from which the lipopolysaccharide is derived or the presence of which is determined is a bacteria selected from the group consisting of Escherichia spp., Pasteurella spp., and Klebsiella spp.

11. The method according to any one of the preceding claims wherein the sample is selected from the group consisting of milk, blood, serum, plasma, urine, saliva, cerebrospinal fluid, peritoneal dialysis fluid, cervical or urethral fluid.

12. The method according to any one of the preceding claims wherein at least one of the at least one capture reagent is selected from the group consisting of a lectin, a lipopolysaccharide binding protein, a lipopolysaccharide binding compound, and an antibiotic.

13. The method according to any one of the preceding claims, wherein the antibiotic is selected from the group consisting of cyclic peptide-comprising antibiotics.

14. The method according to any one of the preceding claims wherein the at least one capture reagent comprises Polymyxin B.

15. The method according to any one of the preceding claims wherein the at least one capture reagent comprises a molecular crowding agent.

16. The method according to any one of the preceding claims wherein the at least one capture reagent comprises a polyethylene or a polyethylene glycol.

17. The method according to any one of the preceding claims wherein the polyethylene glycol is PEG20.

18. The method according to any one of the preceding claims wherein the multimerizing agent is selected from the group consisting of a lectin, a lipopolysaccharide binding protein, a lipopolysaccharide binding compound, and an antibiotic.

19. The method according to any one of the preceding claims wherein the multimerizing agent is a lipopolysaccharide binding protein.

20. The method according to any one of the preceding claims wherein the multimerizing agent is detectably labelled.

21. The method according to any one of the preceding claims wherein the multimerizing agent is conjugated to a detection system.

22. The method according to any one of the preceding claims wherein the multimerizing agent is Concanavalin A.

23. The method according to any one of the preceding claims wherein the method comprises step (d), and wherein the binding reagent of step (d) is an antibody or fragment thereof capable of selectively binding one or more components of the complex comprising lipopolysaccharide analyte and multimeric multimerizing reagent.

24. The method according to any one of the preceding claims wherein the method comprises step (d), and wherein the binding reagent of step (d) is an antibody or fragment thereof having improved binding to a component of the complex comprising lipopolysaccharide analyte and multimeric multimerizing reagent when said component is present in said complex compared to the binding of the binding reagent to the component of the complex when said component is not present in the complex.

25. The method according to any one of the preceding claims, the method comprising the steps of:
(a) a lipopolysaccharide analyte capture step, comprising contacting the sample with a capture reagent comprising Polymyxin B in the presence of a solid substrate; and
(b) contacting the sample with a multimerizing reagent capable of binding to lipopolysaccharide, said multimerizing reagent comprising Concanavalin A and wherein when contacted with the sample at least some of the Concanavalin A is dimeric;
(c) maintaining the lipopolysaccharide analyte and the Concanavilin A in contact for a time and under conditions suitable for multimerization of the Concanavalin A and the formation of a complex comprising lipopolysaccharide analyte and multimeric Concanavalin A, said conditions comprising maintaining the sample at a pH of at least about 7.2;
(d) optionally contacting the sample with binding reagent capable of binding to the complex comprising lipopolysaccharide analyte and multimeric Concanavalin A;
(e) detecting the presence of the complex so formed;
and wherein the presence of the complex is indicative of the presence of a lipopolysaccharide analyte in the sample.

26. The method according to claim 25, wherein prior to contacting the sample in step (b) at least some of the Concanavalin A is maintained at or below pH 5.4.

27. The method according to claim 25 or claim 26 wherein contacting the sample with Concanavalin A comprises raising the pH at which the Concanavalin A is maintained.

28. The method according to any one of claims 25 to 27 wherein contacting the sample with Concanavalin A comprises raising the pH at which the Concanavalin A is maintained to a pH of about 7.2 or above.

29. The method according to any one of the preceding claims, wherein detection of the complex comprises contacting the sample with an antibody-binding reagent.

30. The method according to any one of the preceding claims, wherein the antibody-binding reagent is capable of binding one or more endogenous lipopolysaccharide-binding antibodies present in the sample.

31. The method according to any one of the preceding claims, wherein the method comprises contacting the sample with a binding reagent comprising an antibody or fragment thereof capable of selectively binding the complex comprising lipopolysaccharide analyte.

32. The method according to any one of the preceding claims wherein the method comprises contacting the sample with a binding reagent and the binding reagent comprises polyclonal antibodies, including labelled polyclonal antibodies.

33. The method according to any one of the preceding claims wherein the presence of the binding reagent is determined by contacting the sample with a second binding reagent.

34. The method according to any one of the preceding claims wherein the second binding reagent is an antibody-binding reagent.

35. The method according to any one of the preceding claims wherein at least one of the binding reagents is detectably labelled.

36. The method according to any one of the preceding claims wherein the antibody-binding reagent is an antibody binding protein.

37. The method according to any one of the preceding claims wherein the antibody-binding reagent is selected from the group consisting of: an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

38. The method according to any one of the preceding claims wherein the antibody-binding reagent comprises detectably labelled Protein A or a fragment thereof, and/or detectably labelled Protein G or a fragment thereof.

39. The method according to any one of the preceding claims wherein the presence of the complex comprising lipopolysaccharide and multimeric Concanavalin A is by detection of tetrameric Concanavalin A.

40. The method of claim 39, wherein one or more Concanavalin A monomers or dimers present in the Concanavalin tetramer is labelled with a donor fluorophore, and one or more Concanavalin A monomers or dimers present in the tetramer is labelled with an acceptor fluorophore.

41. The method according to any one of the preceding claims, wherein the method comprises an ELISA.

42. An analytical test device for detecting the presence of a lipopolysaccharide analyte in a liquid biological sample, the device comprising: a solid support having reversibly immobilised thereon in a first zone of the support a capture reagent capable of binding lipopolysaccharide, said capture reagent comprising Polymyxin B, optionally together with one or more molecular crowding reagents.

43. An analytical test device for detecting the presence of a lipopolysaccharide analyte in a liquid biological sample, the device comprising:
(a) a solid support formed of a material enabling capillary flow of fluid along at least a portion of the support;
(b) a sample pad located proximal to one end of the solid support for receiving a sample of the fluid, wherein the sample pad comprises a filter membrane for removal of one or more components from the sample;
(c) a conjugate pad located on or in the solid support so that the sample flows under capillary action along the support from the sample pad to the conjugate pad and mobilises a conjugate contained in the conjugate pad, the conjugate comprising a multimerizing reagent capable of binding lipopolysaccharide, wherein said multimerizing reagent is bound to or capable of being bound by a detection agent;
(d) a detection band comprising a capture reagent immobilised on or in the support along a band located substantially perpendicular to the direction of flow of the sample along the support so that when the mobilised conjugate contacts the capture reagent in the detection band the presence of the lipopolysaccharide analyte in the sample is indicated by a visible colour change or detection of a fluorescence signal; and
(e) optionally a wicking pad for receiving and retaining sample after passing through the detection band.

44. An analytical test device for detecting the presence of a lipopolysaccharide analyte in a liquid biological sample, the device comprising:
(a) a solid support formed of a material enabling capillary flow of liquid along at least a portion of the support;
(b) a sample pad located proximal to one end of the solid support for receiving a sample of the liquid sample, wherein the sample pad comprises one or more buffers, one or more blocking agents, and dimeric Concanavalin A;
(c) a conjugate pad located on or in the solid support so that the liquid sample flows under capillary action along the support from the sample pad to the conjugate pad, wherein the conjugate pad comprises detectably labelled antibody-binding reagent and a buffer system capable of raising the pH of the sample when present at the conjugate pad to at least about 7, wherein the conjugate pad optionally comprises immobilised LPS; and
(d) optionally a wicking pad for receiving and retaining sample after passing through the conjugate pad.

45. The analytical test device according to claim 43 or 44, wherein the sample pad and/or the conjugate pad comprises a reagent selected from the group consisting of PMB, PEG, and immobilised LPS.

46. The analytical test device according to any one of claims 42 to 45, wherein the device comprises an indicator, said indicator comprising bovine IgG as a positive control.

47. The analytical test device according to any one of claims 42 to 46, wherein the detectable label comprises gold particles or gold nanoparticles.

48. A diagnostic kit comprising (a) a capture reagent selected from the group consisting of a lectin, a lipopolysaccharide binding protein, a lipopolysaccharide binding compound, and an antibiotic, (b) a multimerizing reagent capable of binding to lipopolysaccharide;
(c) optionally one or more binding reagents capable of binding to a complex comprising lipopolysaccharide analyte and multimeric multimerizing reagent; and/or
(d) one or more detectably labelled binding reagent capable of binding to a complex comprising lipopolysaccharide analyte and multimeric multimerizing reagent;
(e) optionally one or more reagents for detecting the presence of a labelled reagent;
(f) and instructions for the use of the kit in the detection of one or more lipopolysaccharide analytes in a sample or the presence of gram-negative bacteria in a sample or in a subject from whom a sample has been obtained.

49. The analytical test device of any one of claims 42 to 47 or the diagnostic kit according to claim 48, wherein the capture reagent comprises Polymyxin B.

50. The analytical test device of any one of claims 42 to 47 or 49 or the diagnostic kit according to claim 48 or 49, wherein the multimerizing reagent comprises Concanavalin A.

51. The analytical test device of any one of claims 42 to 47, 49, or 50 or the diagnostic kit according to any one of claims 48 to 50, wherein the detectably labelled binding reagent capable of binding to a complex comprising lipopolysaccharide analyte and multimeric multimerizing reagent is selected from the group consisting of an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

52. A method of treating a disease or condition in a subject or of treating a subject at increased risk of having or developing a disease or condition, wherein the disease or condition is caused by or associated with the presence of a gram-negative bacteria, the method comprising:
(a) providing the results of a diagnostic or prognostic method as contemplated herein;
(b) determining whether a result indicative that the subject has the disease or condition or is at increased risk of developing the disease or condition exists, and identifying a subject accordingly; and
(c) administering a therapeutic regimen to a subject so identified to treat the disease or condition.

53. The method according to claim 52, wherein the therapeutic regimen is treatment with one or more antibiotics, including treatment with one or more antibiotics selected from the group consisting of aminoglycosides, such as gentamicin, amikacin; beta-lactams; carbapenems, including imipenem, meropenem; cephalosporins, including cefotaxime, ceftazidime; chloramphenicols; fluorquinolones, such as ciprofloxacin, delafloxacin; fosfomycin; penicillins; polymyxins, such as colistin, polymyxin B; glycylcycline, such as tigecycline; sulphonamides, such as co-trimoxazole; tetracyclines, including doxycycline, eravacycline, minocycline, omadacycline; and ureidopenicillins, such as piperacillin; and any combination of two or more thereof, including combinations such as ceftolozane/tazobactam, ceftazidime/avibactam, meropenem/vaborbactam, and imipenem/cilastatin/relebactam.
